(19) United States
(12) Patent Application Publication
(10) Pub. No.: US 2025/0011833 A1
(43) Pub. Date: Jan. 9, 2025

(54) METHODS AND COMPOSITIONS FOR SEQUENCING AND FUSION DETECTION USING RANDOMER REVERSE PRIMERS

(71) Applicants: William Marsh Rice University, Houston, TX (US); NuProbe USA, Inc., Houston, TX (US)

(72) Inventors: Ping SONG, Houston, TX (US); David Y. ZHANG, Houston, TX (US); Wenjun LI, Houston, TX (US)

(73) Assignees: William Marsh Rice University, Houston, TX (US); NuProbe USA, Inc., Houston, TX (US)

(21) Appl. No.: 18/708,952

(22) PCT Filed: Nov. 9, 2022
(86) PCT No.: PCT/US2022/079539

§ 371 Date: May 9, 2024

Related U.S. Application Data
(60) Provisional application No. 63/277,552, filed on Nov. 9, 2021.

(51) Int. Cl. C12Q 1/6806; C12N 15/10

(52) U.S. Cl. CPC C12Q 1/6806; C12N 15/1093

(57) ABSTRACT

This disclosure provides methods and compositions comprising random reverse primers that are useful for the amplification of nucleic acid molecules.

### CROSS-REFERENCE TO RELATED APPLICATION AND INCORPORATION OF SEQUENCE LISTING

This application claims the benefit of U.S. Provisional Application No. 63/277,552, filed Nov. 9, 2021, which is incorporated by reference in its entirety herein. A sequence listing contained in the file named “P35050WO00_SL.XML” which is 56,488 bytes (measured in MS-Windows®) and created on Nov. 8, 2022, is filed electronically herewith and incorporated by reference in its entirety.

### FIELD

The present disclosure relates to the field of molecular biology. More particularly, it relates to methods and compositions useful for the amplification of nucleic acid molecules.

### INCORPORATION OF SEQUENCE LISTING

Table 1 provides nucleic acid sequences used in this application.

TABLE 1
Nucleic acid sequences

| SEQ | | Sequence | |
|---|---|---|---|
| ID NO. | Description | Type | Sequence |
| 1 | RRP-RS11 | Nucleic | GTCTCGTGGGCTCGGAGATGTGTATAAGAGACAGGTGC |
| | | acid | AAGNNNNNNNNNNN |
| 2 | Adapter_2 | Nucleic | GTCTCGTGGGCTCGGAGATGTGTATAAGAGACAG |
| | | acid | |
| 3 | Adapter_3 | Nucleic | CGTGGGCTCGGAGATGTG |
| | | acid | |
| 4 | Uni_long_OFP | Nucleic | TGCGATGCAATGAGAACATTAGAAGTCAGCTGCATGAC |
| | (same as | acid | TGAAGA |
| | Adapter_1) | | |
| 5 | OFP-1_NTRK | Nucleic | TGCGATGCAATGAGAACATTAGAAGTCAGCTGCATGAC |
| | | acid | TGAAGAGGGGATGCACCAGTGGT |
| 6 | OFP-2_NTRK | Nucleic | TGCGATGCAATGAGAACATTAGAAGTCAGCTGCATGAC |
| | | acid | TGAAGAAAAGGTGTTTCGTCCTTCTTCTC |
| 7 | OFP-3_NTRK | Nucleic | TGCGATGCAATGAGAACATTAGAAGTCAGCTGCATGAC |
| | | acid | TGAAGAAAAGGAAGAGGCAGGCAAAGA |
| 8 | OFP-4_NTRK | Nucleic | TGCGATGCAATGAGAACATTAGAAGTCAGCTGCATGAC |
| | | acid | TGAAGATGTCATGAAATGCAGGGACATG |
| 9 | OFP-5_NTRK | Nucleic | TGCGATGCAATGAGAACATTAGAAGTCAGCTGCATGAC |
| | | acid | TGAAGAAGCTCCCACTTGAGCACG |
| 10 | IFP-1_NTRK | Nucleic | TCGTCGGCAGCGTCAGATGTGTATAAGAGACAGCCACC |
| | | acid | GCCGTGTGCAG |
| 11 | IFP-2_NTRK | Nucleic | TCGTCGGCAGCGTCAGATGTGTATAAGAGACAGCCGGG |
| | | acid | TCTCCAGATGTGC |
| 12 | IFP-3_NTRK | Nucleic | TCGTCGGCAGCGTCAGATGTGTATAAGAGACAGGGCCA |
| | | acid | GGCCCACAGC |
| 13 | IFP-4_NTRK | Nucleic | TCGTCGGCAGCGTCAGATGTGTATAAGAGACAGCCCAT |
| | | acid | CCTCTGGAGCCAG |
| 14 | IFP-5_NTRK | Nucleic | TCGTCGGCAGCGTCAGATGTGTATAAGAGACAGGTCCC |
| | | acid | GGCGCTTGATGT |
| 15 | gBlock-1_NTRK | Nucleic | CTTCAAGGCAGAGCAAATGTGACACTGTATCAGTACTG |
| | | acid | GAAGAAGACATTCATACATTAAGGCATGGAAGTGAATA |
| | | | AGCGGGCCGGGCAGAGGTCTCTGTTCAGGTCAACGTCT |
| | | | CCTTCCCGGCCAGTGTGCAGCTGCACACGGCGGTGGAG |
| | | | ATGCACCACTGGTGCATCCCCTTCTCTGTGGATGGGCA |
| | | | GCCGGCACCGTCTCTGCGCTGGCTCTTCAATGGCTCCG |
| | | | TGCTCAATGAGACCAGCTTCATCTTCACTGAGTTCCTG |
| | | | GAGCCGGCAGCCAATGAGACCGTGCGGCACGGGTGTCT |
| | | | GCGCCTCAACCAGCCCACCCACGTCAACAACGGCAACT |
| | | | ACACGCTGCTGGCTGCCAACCCCTTCGGCCAGGCCTCC |
| | | | GCCTCCATCATGGCTGCCTTCATGGACAACCCTTTCGA |
| | | | GTTCAACCCCGAGGACCCCATCCCTGACACTAACAGCA |
| | | | CATCTGGAGACCCGGTGGAGAAGAAGGACGAAACACCT |
| | | | TTTGGG |
| 16 | gBlock-2_NTRK | Nucleic | CTTCAAGGCAGAGCAAATGTGACACTGTATCAGTACTG |
| | | acid | GAAGAAGACATTCATACATTAAGGCATGGAAGTGAATA |
| | | | AGCGATGGGCAGCCGGCACCGTCTCTGCGCTGGCTCTT |
| | | | CAATGGCTCCGTGCTCAATGAGACCAGCTTCATCTTCA |
| | | | CTGAGTTCCTGGAGCCGGCAGCCAATGAGACCGTGCGG |
| | | | CACGGGTGTCTGCGCCTCAACCAGCCCACCCACGTCAA |
| | | | CAACGGCAACTACACGCTGCTGGCTGCCAACCCCTTCG |
| | | | GCCAGGCCTCCGCCTCCATCATGGCTGCCTTCATGGAC |
| | | | AACCCTTTCGAGTTCAACCCCGAGGACCCCATCCCTGA |
| | | | CACTAACAGCACATCTGGAGACCCGGTGGAGAAGAAGG |
| | | | ACGAAACACCTTTTGGGGTCTCGGTGGCTGTGGGCCTG |
| | | | GCCGTCTTTGCCTGCCTCTTCCTTTCTACGCTGCTCCT |
| | | | TGTGCTCAACAAATGTGGACGGAGAAACAAGTTTGGGA |
| | | | TCAACC |
| 17 | gBlock-3_NTRK | Nucleic | CTTCAAGGCAGAGCAAATGTGACACTGTATCAGTACTG |
| | | acid | GAAGAAGACATTCATACATTAAGGCATGGAAGTGAATA |
| | | | AGCGGGAGACCCGGTGGAGAAGAAGGACGAAACACCTT |
| | | | TTGGGGTCTCGGTGGCTGTGGGCCTGGCCGTCTTTGCC |
| | | | TGCCTCTTCCTTTCTACGCTGCTCCTTGTGCTCAACAA |
| | | | ATGTGGACGGAGAAACAAGTTTGGGATCAACCGCCCGG |
| | | | CTGTGCTGGCTCCAGAGGATGGGCTGGCCATGTCCCTG |
| | | | CATTTCATGACATTGGGTGGCAGCTCCCTGTCCCCCAC |
| | | | CGAGGGCAAAGGCTCTGGGCTCCAAGGCCACATCATCG |
| | | | AGAACCCACAATACTTCAGTGATGCCTGTGTTCACCAC |
| | | | ATCAAGCGCCGGGACATCGTGCTCAAGTGGGAGCTGGG |
| | | | GGAGGGCGCCTTTGGGAAGGTCTTCCTTGCTGAGTGCC |
| | | | ACAACCTCCTGCCTGAGCAGGACAAGATGCTGGTGGCT |
| | | | GTCAAG |
| 18 | gBlock-4_NTRK | Nucleic | CTTCAAGGCAGAGCAAATGTGACACTGTATCAGTACTG |
| | | acid | GAAGAAGACATTCATACATTAAGGCATGGAAGTGAATA |
| | | | AGCGCATCCCTGCTGTCCACCCGGGTTCATTCAGGCCC |
| | | | AAGTGCGACGAGAACGGCAACTATCTGCCACTCCAGTG |
| | | | CTATGGGAGCATCGGCTACTGCTGGTGTGTCTTCCCCA |
| | | | ACGGCACGGAGGTCCCCAACACCAGAAGCCGCGGGCAC |
| | | | CATAACTGCAGTGAGTCACTGGAACTGGAGGACCCGTC |
| | | | TTCTGGGCTGGGTGTGACCAAGCAGGATCTGGGCCCAG |
| | | | ACACTAACAGCACATCTGGAGACCCGGTGGAGAAGAAG |
| | | | GACGAAACACCTTTTGGGGTCTCGGTGGCTGTGGGCCT |
| | | | GGCCGTCTTTGCCTGCCTCTTCCTTTCTACGCTGCTCC |
| | | | TTGTGCTCAACAAATGTGGACGGAGAAACAAGTTTGGG |
| | | | ATCAACCGCCCGGCTGTGCTGGCTCCAGAGGATGGGCT |
| 19 | gBlock-5_NTRK | Nucleic | CTTCAAGGCAGAGCAAATGTGACACTGTATCAGTACTG |
| | | acid | GAAGAAGACATTCATACATTAAGGCATGGAAGTGAATA |
| | | | AGCGTTAAGGAGCTGAAAGCGCGCAATACCAAGAAGGA |
| | | | GGGTGACCTGATAGCTGCTCAGGCTCGGCTGAAGGACC |
| | | | TGGAGGCTCTGCTGAACTCCAAGGAGGCCGCACTGAGC |
| | | | ACTGCTCTCAGTGAGAAGCGCACGCTGGAGGGCGAGCT |
| | | | GCATGATCTGCGGGGCCAGGTGGCCAAGGTCTCGGTGG |
| | | | CTGTGGGCCTGGCCGTCTTTGCCTGCCTCTTCCTTTCT |
| | | | ACGCTGCTCCTTGTGCTCAACAAATGTGGACGGAGAAA |
| | | | CAAGTTTGGGATCAACCGCCCGGCTGTGCTGGCTCCAG |
| | | | AGGATGGGCTGGCCATGTCCCTGCATTTCATGACATTG |
| | | | GGTGGCAGCTCCCTGTCCCCCACCGAGGGCAAAGGCTC |
| | | | TGGGCTCCAAGGCCACATCATCGAGAACCCACAATACT |
| | | | TCAGT |
| 20 | gBlock-6_NTRK | Nucleic | CTTCAAGGCAGAGCAAATGTGACACTGTATCAGTACTG |
| | | acid | GAAGAAGACATTCATACATTAAGGCATGGAAGTGAATA |
| | | | AGCGCTACCGACCTGGTGTGGAAGGCACAGAACACCTG |
| | | | GGGCTGCGGGAACAGCCTGCGTACGGCTCTCATCAACT |
| | | | CCACTGGGGAAGAAGTGGCCATGCGCAAGCTGGTGCGC |
| | | | TCAGTGACTGTGGTTGAGGACGACGAGGATGAGGATGG |
| | | | AGATGACCTGCTCCATCACCACCACGTCTCGGTGGCTG |
| | | | TGGGCCTGGCCGTCTTTGCCTGCCTCTTCCTTTCTACG |
| | | | CTGCTCCTTGTGCTCAACAAATGTGGACGGAGAAACAA |
| | | | GTTTGGGATCAACCGCCCGGCTGTGCTGGCTCCAGAGG |
| | | | ATGGGCTGGCCATGTCCCTGCATTTCATGACATTGGGT |
| | | | GGCAGCTCCCTGTCCCCCACCGAGGGCAAAGGCTCTGG |
| | | | GCTCCAAGGCCACATCATCGAGAACCCACAATACTTCA |
| | | | GT |
| 21 | gBlock-7_NTRK | Nucleic | CTTCAAGGCAGAGCAAATGTGACACTGTATCAGTACTG |
| | | acid | GAAGAAGACATTCATACATTAAGGCATGGAAGTGAATA |
| | | | AGCGCTACCGACCTGGTGTGGAAGGCACAGAACACCTG |
| | | | GGGCTGCGGGAACAGCCTGCGTACGGCTCTCATCAACT |
| | | | CCACTGGGGAAGAAGTGGCCATGCGCAAGCTGGTGCGC |
| | | | TCAGTGACTGTGGTTGAGGACGACGAGGATGAGGATGG |
| | | | AGATGACCTGCTCCATCACCACCACGCCCGGCTGTGCT |
| | | | GGCTCCAGAGGATGGGCTGGCCATGTCCCTGCATTTCA |
| | | | TGACATTGGGTGGCAGCTCCCTGTCCCCCACCGAGGGC |
| | | | AAAGGCTCTGGGCTCCAAGGCCACATCATCGAGAACCC |
| | | | ACAATACTTCAGTGATGCCTGTGTTCACCACATCAAGC |
| | | | GCCGGGACATCGTGCTCAAGTGGGAGCTGGGGGAGGGC |
| | | | GCCTTTGGGAAGGTCTTCCTTGCTGAGTGCCACAAC |
| 22 | IFP-1_FGFR2 | Nucleic | TCGTCGGCAGCGTCAGATGTGTATAAGAGACAGGCCAT |
| | | acid | TCAAGGTAATCGCCG |
| 23 | IFP-2_FGFR2 | Nucleic | TCGTCGGCAGCGTCAGATGTGTATAAGAGACAGCTTCA |
| | | acid | GTTTAGTTGAGGATACCACAT |
| 24 | IFP-3_FGFR2 | Nucleic | TCGTCGGCAGCGTCAGATGTGTATAAGAGACAGGAAAC |
| | | acid | TTGGTACTTCATGGTGAATG |
| 25 | IFP-4_FGFR2 | Nucleic | TCGTCGGCAGCGTCAGATGTGTATAAGAGACAGGAAGA |
| | | acid | TTTTGTCAGTGAGAACAGTAAC |
| 26 | IFP-5_FGFR2 | Nucleic | TCGTCGGCAGCGTCAGATGTGTATAAGAGACAGCAGGA |
| | | acid | GCATCGCATTGGAG |
| 27 | IFP-6_FGFR2 | Nucleic | TCGTCGGCAGCGTCAGATGTGTATAAGAGACAGCAATC |
| | | acid | ACACGTACCACCTGGAT |
| 28 | IFP-7_FGFR2 | Nucleic | TCGTCGGCAGCGTCAGATGTGTATAAGAGACAGGGGCT |
| | | acid | GCCCTACCTCAAG |
| 29 | IFP-8_FGFR2 | Nucleic | TCGTCGGCAGCGTCAGATGTGTATAAGAGACAGCTCAC |
| | | acid | TGTCCTGCCAAAACAG |
| 30 | IFP-9_FGFR2 | Nucleic | TCGTCGGCAGCGTCAGATGTGTATAAGAGACAGCTCTG |
| | | acid | CATGGTTGACAGTTC |
| 31 | IFP-10_FGFR2 | Nucleic | TCGTCGGCAGCGTCAGATGTGTATAAGAGACAGCCAAA |
| | | acid | CGTATCCCCCTGCG |
| 32 | IFP-11_FGFR2 | Nucleic | TCGTCGGCAGCGTCAGATGTGTATAAGAGACAGGACCC |
| | | acid | AAAATGGGAGTTTCCAA |
| 33 | IFP-12_FGFR2 | Nucleic | TCGTCGGCAGCGTCAGATGTGTATAAGAGACAGACCGT |
| | | acid | GGCCGTGAAGATG |
| 34 | IFP-13_FGFR2 | Nucleic | TCGTCGGCAGCGTCAGATGTGTATAAGAGACAGATAAA |
| | | acid | TCTTCTTGGAGCCTGCAC |
| 35 | IFP-14_FGFR2 | Nucleic | TCGTCGGCAGCGTCAGATGTGTATAAGAGACAGGGCAT |
| | | acid | GGAGTACTTGGCTTC |
| 36 | IFP-15_FGFR2 | Nucleic | TCGTCGGCAGCGTCAGATGTGTATAAGAGACAGGCCAG |
| | | acid | AGATATCAACAATATAGACTATTACA |
| 37 | IFP-16_FGFR2 | Nucleic | TCGTCGGCAGCGTCAGATGTGTATAAGAGACAGCCTGT |
| | | acid | TTGATAGAGTATACACTCATCAGA |
| 38 | IFP-17_FGFR2 | Nucleic | TCGTCGGCAGCGTCAGATGTGTATAAGAGACAGGCCAG |
| | | acid | CCAACTGCACCA |
| 39 | IFP-18_FGFR2 | Nucleic | TCGTCGGCAGCGTCAGATGTGTATAAGAGACAGGGATC |
| | | acid | GAATTCTCACTCTCACAAC |
| 40 | OFP-1_FGFR2 | Nucleic | TGCGATGCAATGAGAACATTAGAAGTCAGCTGCATGAC |
| | | acid | TGAAGACGGAGGAGCGTTGCCAT |
| 41 | OFP-2_FGFR2 | Nucleic | TGCGATGCAATGAGAACATTAGAAGTCAGCTGCATGAC |
| | | acid | TGAAGACCTGGCCCGGCCCT |
| 42 | OFP-3_FGFR2 | Nucleic | TGCGATGCAATGAGAACATTAGAAGTCAGCTGCATGAC |
| | | acid | TGAAGATACTGCCAGTAGGACTGTAGACA |
| 43 | OFP-4_FGFR2 | Nucleic | TGCGATGCAATGAGAACATTAGAAGTCAGCTGCATGAC |
| | | acid | TGAAGAATGAGGATGACACCGATGGTG |
| 44 | OFP-5_FGFR2 | Nucleic | TGCGATGCAATGAGAACATTAGAAGTCAGCTGCATGAC |
| | | acid | TGAAGAGCTGAAAAACGGGAAGGAGTTTA |
| 45 | OFP-6_FGFR2 | Nucleic | TGCGATGCAATGAGAACATTAGAAGTCAGCTGCATGAC |
| | | acid | TGAAGATGTAGTGGAGAATGAATACGGGTC |
| 46 | OFP-7_FGFR2 | Nucleic | TGCGATGCAATGAGAACATTAGAAGTCAGCTGCATGAC |
| | | acid | TGAAGAGGAAAAGAACGGCAGTAAATACGG |
| 47 | OFP-8_FGFR2 | Nucleic | TGCGATGCAATGAGAACATTAGAAGTCAGCTGCATGAC |
| | | acid | TGAAGAAGGCCAACCAGTCTGCC |
| 48 | OFP-9_FGFR2 | Nucleic | TGCGATGCAATGAGAACATTAGAAGTCAGCTGCATGAC |
| | | acid | TGAAGAGTGCTTGGCGGGTAATTCTATTG |
| 49 | OFP-10_FGFR2 | Nucleic | TGCGATGCAATGAGAACATTAGAAGTCAGCTGCATGAC |
| | | acid | TGAAGAAGCCAGACTTCAGCAGCCA |
| 50 | OFP-11_FGFR2 | Nucleic | TGCGATGCAATGAGAACATTAGAAGTCAGCTGCATGAC |
| | | acid | TGAAGAGTCTCCGAGTATGAACTTCCAGA |
| 51 | OFP-12_FGFR2 | Nucleic | TGCGATGCAATGAGAACATTAGAAGTCAGCTGCATGAC |
| | | acid | TGAAGAACAAGCCCAAGGAGGCG |
| 52 | OFP-13_FGFR2 | Nucleic | TGCGATGCAATGAGAACATTAGAAGTCAGCTGCATGAC |
| | | acid | TGAAGAATGAAGATGATTGGGAAACACAAGAATAT |
| 53 | OFP-14_FGFR2 | Nucleic | TGCGATGCAATGAGAACATTAGAAGTCAGCTGCATGAC |
| | | acid | TGAAGATGGTGTCATGCACCTACCAG |
| 54 | OFP-15_FGFR2 | Nucleic | TGCGATGCAATGAGAACATTAGAAGTCAGCTGCATGAC |
| | | acid | TGAAGATGATGAAAATAGCAGACTTTGGACTC |
| 55 | OFP-16_FGFR2 | Nucleic | TGCGATGCAATGAGAACATTAGAAGTCAGCTGCATGAC |
| | | acid | TGAAGAAAGTGGATGGCTCCAGAAGC |
| 56 | OFP-17_FGFR2 | Nucleic | TGCGATGCAATGAGAACATTAGAAGTCAGCTGCATGAC |
| | | acid | TGAAGACTGAAGGAAGGACACAGAATGGATA |
| 57 | OFP-18_FGFR2 | Nucleic | TGCGATGCAATGAGAACATTAGAAGTCAGCTGCATGAC |
| | | acid | TGAAGACGTTCAAGCAGTTGGTAGAAGAC |

### BACKGROUND

About 20% of diseases are related to gene fusion variants. Gene fusion, including RNA fusion and DNA fusion, comprises known fusion and unknown fusion. Known fusion refers to a situation in which all fusion partners are known. Unknown fusion refers to the situation in which only one partner is known, and other partners are unknown. For known fusions, researching usually rely on ligation with an adapter or design primers based on the partners to amplify the known fusion. For unknown fusions, since only one partner of the fusion is known, only one gene-specific primer can be designed based on the known partner. To amplify the unknown partner(s), a ligation method can be used to add a universal adapter. The ligation method is limited by low ligation yield.

RNA splicing variants include exon skipping, alternative 5′ and 3′ splice sites, mutually exclusive exons, intron retention, and alternative splicing coupled with alternative first or last exons. Current RNA splicing variant detection methods include ligation-based sequencing and capture probe based pull down panels. However, there are numerous modes of the alternative splicing variants, and using ligation-based methods or capture-based methods to obtain specific RNA splicing variants remains a challenge.

### SUMMARY

In one aspect, this disclosure provides a method of preparing a sequencing library, the method comprising: (a) introducing to a sample comprising at least one nucleic acid template molecule: (i) at least one Randomer Reverse Primer (RRP), where the at least one RRP comprises a Randomer Sequence (RS) positioned within the 3′ region of the at least one RRP and a first universal adapter sequence positioned 5′ to the RS: (ii) a first polymerase; and (iii) a reaction buffer to create a first mixture: (b) subjecting the first mixture to a first polymerase extension step to generate a second mixture comprising at least one extended product: (c) introducing to the second mixture: (i) at least one Inner Forward Primer (IFP) that binds to an IFP binding site on the at least one nucleic acid template molecule: (ii) a second adapter sequence: (iii) a thermostable polymerase; and (iv) reagents for thermostable polymerase activity to generate a third mixture; and (d) subjecting the third mixture to a second polymerase extension step to generate at least one amplicon of the at least one nucleic acid template molecule.

In one aspect, this disclosure provides a method of preparing a sequencing library, the method comprising: (a) introducing to a sample comprising at least one nucleic acid template molecule: (i) at least one Randomer Reverse Primer (RRP), where the at least one RRP comprises a Randomer Sequence (RS) positioned on the 3′ region of the at least one RRP and a first universal adapter sequence positioned 5′ to the RS: (ii) at least one Outer Forward Primer (OFP): (iii) a first polymerase; and (iv) a reaction buffer to create a first mixture: (b) subjecting the first mixture to a first polymerase extension step to generate a second mixture comprising at least one extended product: (c) introducing to the second mixture: (i) at least one Inner Forward Primer (IFP) that binds to an IFP binding site on the at least one nucleic acid template molecule: (ii) a second adapter sequence: (iii) a thermostable polymerase, and (iv) reagents for thermostable polymerase activity to generate a third mixture; and (d) subjecting the third mixture to a second polymerase extension step to generate at least one amplicon of the at least one nucleic acid template molecule.

In one aspect, this disclosure provides a method for detecting at least one gene fusion in a test sample, the method comprising: (a) obtaining DNA extracted from a cell line or a clinical patient sample, or obtaining cDNA generated from RNA extracted from a cell line or a clinical patent sample to generate the test sample: (b) introducing to the test sample: (i) at least one Randomer Reverse Primer (RRP), where the at least one RRP comprises a Randomer Sequence (RS) positioned on the 3′ end of the at least one RRP and a first universal adapter sequence positioned 5′ to the RS: (ii) at least one Outer Forward Primer (OFP) that binds to an OFP binding site: (iii) a first polymerase; and (iv) a reaction buffer to create a first mixture: (c) subjecting the first mixture to a first polymerase extension step to generate at least one extended product: (d) diluting or purifying the at least one extended product to generate a second mixture comprising the at least one extended product: (e) introducing to the second mixture (i) at least one Inner Forward Primer (IFP) that binds to an IFP binding site: (ii) a universal reverse primer: (iii) a thermostable polymerase; and (iv) reagents for thermostable polymerase activity to generate a third mixture: (f) subjecting the third mixture to a second polymerase extension step to generate at least one amplicon from the at least one extended product; and (g) analyzing the at least one amplicon to identify an amplicon comprising the at least one gene fusion.

In an aspect, this disclosure provides a method of designing a plurality of Inner Forward Primers (IFPs) and a plurality of Outer Forward Primers (OFPs) to identify a gene fusion, within the gene of interest, where an adjacent IFP and OFP designed to amplify the same target region of the gene of interest form a primer set, and where: (a) the 3′ end of an OFP binding site and the 5′ end of an IFP binding site overlap for a primer set: (b) at least one nucleotide is positioned between the 3′ end of an OFP binding site and the 5′ end of an IFP binding site for a primer set: or (c) zero nucleotides are positioned between the 3′ end of an OFP binding site and the 5′ end of the IFP binding site, and the OFP binding site and an IFP binding site do not overlap for a primer set: where the IFPs and OFPs are used to identify a gene fusion in the nucleic acid molecule.

In an aspect, this disclosure provides A method for detecting alternative RNA splicing, the method comprising: (a) introducing to a sample comprising cDNA generated from an RNA sample comprising at least one RNA splicing variant: (i) at least one Randomer Reverse Primer (RRP), where the at least one RRP comprises a Randomer Sequence (RS) positioned on the 3′ end of the at least one RRP and a first universal adapter sequence positioned 5′ to the RS: (ii) a first polymerase; and (iii) a reaction buffer to create a first mixture: (b) subjecting the first mixture to a first polymerase extension step to generate a second mixture comprising at least one extended product: (c) purifying the at least one extended product from step (b) to generate a second mixture comprising the at least one extended product: (d) introducing to the second mixture: (i) at least one Inner Forward Primer (IFP) that binds to an IFP binding site on the at least one nucleic acid template molecule: (ii) a second adapter sequence: (iii) a thermostable polymerase; and (iv) reagents for thermostable polymerase activity to generate a third mixture: (e) subjecting the third mixture to thermal cycling to generate at least one amplicon of the at least one extended product; and (f) analyzing the at least one amplicon to identify the at least one RNA splicing variant.

### BRIEF DESCRIPTION OF THE DRAWINGS

FIG. 1 depicts a schematic for producing outer PCR amplicons. The DNA template obtains outer polymerase extension using an Outer Forward Primer (OFP) and a Randomer Reverse Primer (RRP).

FIG. 2 depicts a schematic for producing outer PCR amplicons using two steps of outer PCR. The first step uses an Outer Forward Primer (OFP) that is a Gene Specific OFP (GSOFP) in conjunction with a Randomer Reverse Primer (RRP), which comprises a Randomer Sequence (RS). The second step uses a Universal Long Outer Forward Primer (ULOFP) in conjunction with a Universal Long Reverse Primer (ULRP). NRS refers to a Non-Relevant Sequence.

FIG. 3 depicts a schematic for producing outer PCR amplicons using a non-thermostable polymerase. A DNA template is denatured into single stranded DNA before being mixed with a Randomer Reverse Primer (RRP) and a non-thermostable DNA polymerase to allow isothermal extension to occur. Following isothermal extension, the extension products are subjected to outer PCR using Outer Forward Primers (OFP) and Universal Long Outer Forward Primers (Univ_long_OFP).

FIG. 4 depicts a schematic for producing nested amplicons using an Inner AD-FP (an Inner Forward Primer with an Adapter) and an Adapter_3 primer.

FIG. 5 depicts Nested Amplicons that undergo Index PCR to add an index primer for next generation sequencing to the amplicons.

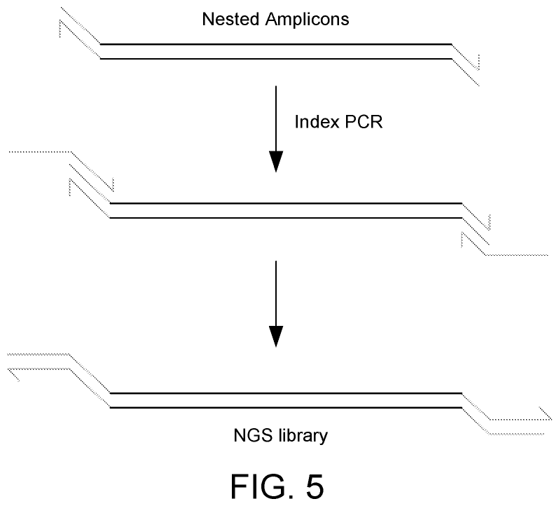

FIG. 6 depicts structures of Randomer Reverse Primers (RRP). FIG. 6a depicts an RRP comprising, from 5′ to 3′, an Adapter and a Randomer Sequence (RS) comprising x nucleotides. FIG. 6b depicts an RRP comprising, from 5′ to 3′, an Adapter, a Non-Relevant Sequence (NRS) of x nucleotides, and an RS of x nucleotides. FIG. 6c depicts an RRP comprising, from 5′ to 3′, an Adapter, a Unique Molecular Identifier (UMI), an NRS of x nucleotides, and an RS of x nucleotides. FIG. 6d depicts an RRP comprising, from 5′ to 3′, an Adapter, an RS of x nucleotides, and a Mixture of Specific Sequence (MSS) of x nucleotides. FIG. 6e depicts an RRP comprising, from 5′ to 5′, an Adapter, an NRS of x nucleotides, an RS of x nucleotides, and an MSS of x nucleotides. FIG. 6f depicts an RRP comprising, from 5′ to 3′, an Adapter, a UMI, an NRS of x nucleotides, an RS of x nucleotides, and an MSS of ex nucleotides. FIG. 6g depicts an RRP comprising, from 5′ to 3′, a hairpin structure and an RS of x nucleotides.

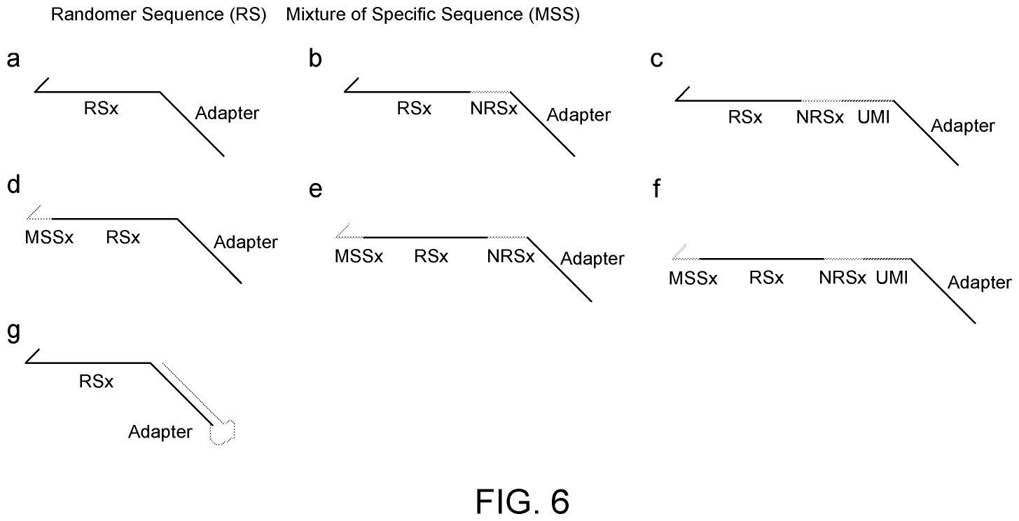

FIG. 7 depicts potential binding positions for Gene Specific Outer Forward Primers (GSOFP) and Gene Specific Inner Forward Primers (GSIFP) on a nucleic acid template molecule. FIG. 7a depicts a tiled arrangement. FIG. 7b depicts a gapped arrangement. FIG. 7c depicts an overlapped arrangement.

FIG. 8 depicts a workflow for designing Gene Specific Outer Forward Primers (GSOFP) and Gene Specific Inner Forward Primers (GSIFP).

FIG. 9 depicts a design scheme for detecting an unknown DNA fusion. FIG. 9a depicts an unknown gene fusion where Gene 1 is known, and is upstream (5′) to the unknown Gene 2. Inner Forward Primers (IFP) and Outer Forward Primers (OFP) are designed to tile Gene 1 Intron 2 to identify the gene fusion. FIG. 9b depicts a strategy to design IFP and OFP primers to the negative strand of DNA when the downstream (3′) member of the fusion is known.

FIG. 10 depicts the repeatability of using Randomer Reverse Primers. Results from two identical experiments are shown using five-plex Inner Forward Primers and Outer Forward Primers to target the NTRK1 gene from exon 7 to exon 11.

FIG. 11 depicts results from using Randomer Reverse Primers (RRP) using a non-thermostable polymerase. FIG. 11a depicts a schematic showing a Gene Specific Inner Forward Primer (GSIFP) binding position in exon 7 of the NTRK1 gene. FIG. 11b depicts the correct start reads of five different Inner Forward Primer loci. FIG. 11c depicts gene fusion variant mimicking gBlocks that were detected using the RRP approach. Five gBlocks were split into two groups based on two reference wildtype templates.

FIG. 12 depicts the use of Randomer Reverse Primers targeting the FGFR2 gene using human brain mRNA. FIG. 12a and FIG. 12b depict bioanalyzer traces of the final libraries used. FIG. 12c depicts the number of correct start reads for each locus sorted by one repeat. FIG. 12d depicts the number of correct start reads and correct start rate of two repeats of the 18-plex FGFR2 panel.

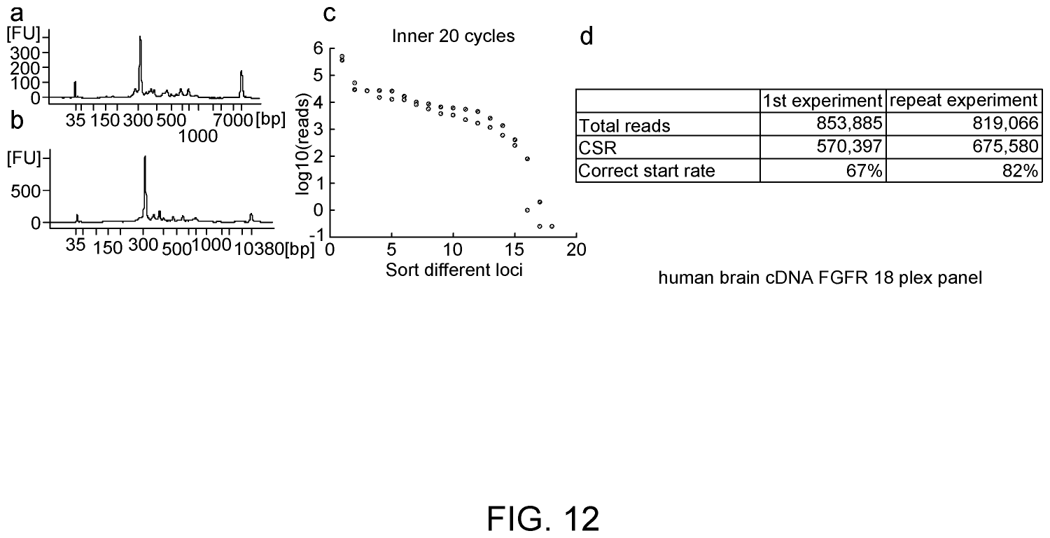

FIG. 13 depicts a schematic combining Blocker Displacement Amplification (BDA) with a Randomer Reverse Primer (RRP) approach to amplify targets. When the template is wildtype, the blocker will bind to the template and the Gene Specific Inner Forward Primer (GSIFP) will be displaced by the blocker so that the wildtype template cannot be efficiently amplified. If the template is a fusion variant, the blocker binding to the template is not favorable thermodynamically. Then, the GSIFP can displace the blocker so that the PCR amplification has higher efficiency. After BDA PCR, the inner PCR is conducted with inner AD-FP (an Inner Forward Primer with an Adapter) to add the adapter to obtain Nested Amplicons.

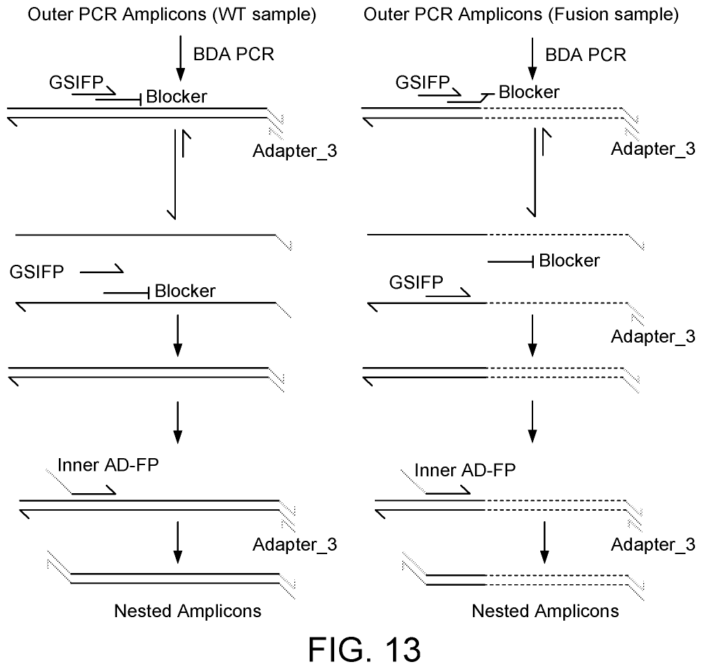

FIG. 14 depicts next generation sequencing data comparing (1) two cycles of outer PCR followed by purification and 18 additional cycles of outer PCR and (2) isothermal extension followed by 20 cycles of outer PCR. FIG. 14a depicts the results from two replicates of the protocol using two cycles of outer PCR followed by purification and 18 additional cycles of outer PCR. FIG. 14b depicts the Randomer Reverse Primer binding position defined by Non-Relevant Sequence (NRS) binding position in Read 1. FIG. 14c depicts the results from two replicates of the protocol using isothermal extension followed by 20 cycles of outer PCR. FIG. 14d depicts the Randomer Reverse Primer binding position defined by NRS binding position in Read 1. There are more binding positions as compared to FIG. 14b.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

Any composition provided herein is specifically envisioned for use with any applicable method provided herein.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D: A and C: B and C: etc.

When a range of numbers is provided herein, the range is understood to inclusive of the edges of the range as well as any number between the defined edges of the range. For example, “between 1 and 10” includes any number between 1 and 10, as well as the number 1 and the number 10.

As used herein, the singular form “a,” “an,” and “the” include plural references unless the context clearly dictates otherwise. For example, the term “a compound” or “at least one compound” may include a plurality of compounds, including mixtures thereof. As used herein, the term “plurality” refers to any whole number greater than one.

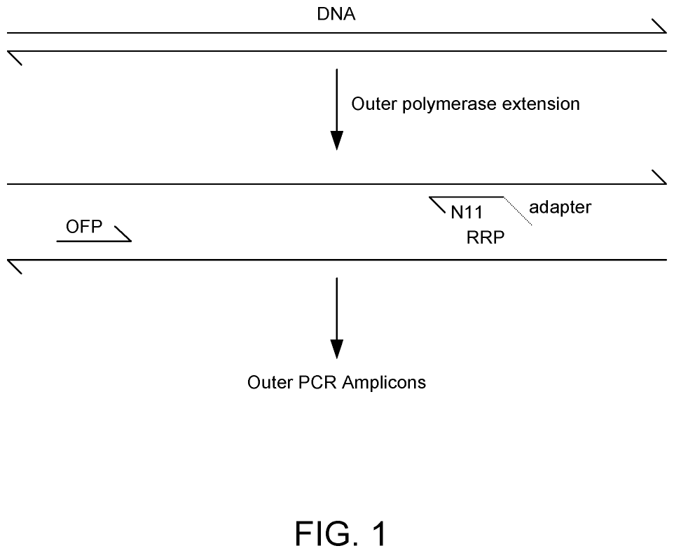

The Randomer Reverse Primer (RRP) approaches provided herein use a primer pair including gene-specific primers and randomer reverse primers. See FIG. 1. The RRP can bind to any position in the downstream portion of a target gene/locus, while the gene-specific primer acts as a forward primer. These methods are based on PCR methods, which have a higher conversion yield compared to ligation-based methods. Using a gene-specific primer will enrich for a specific gene, and using an RRP allows amplification even when the other side of the target gene/locus is unknown.

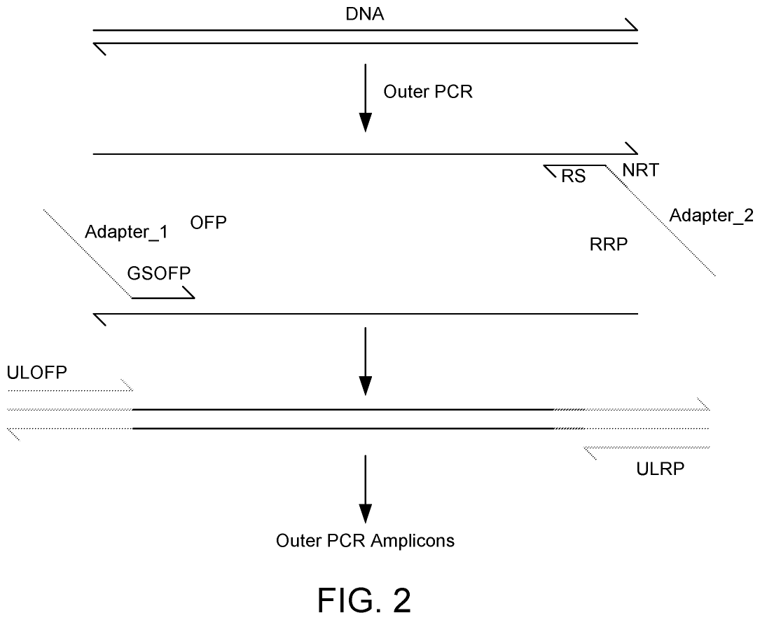

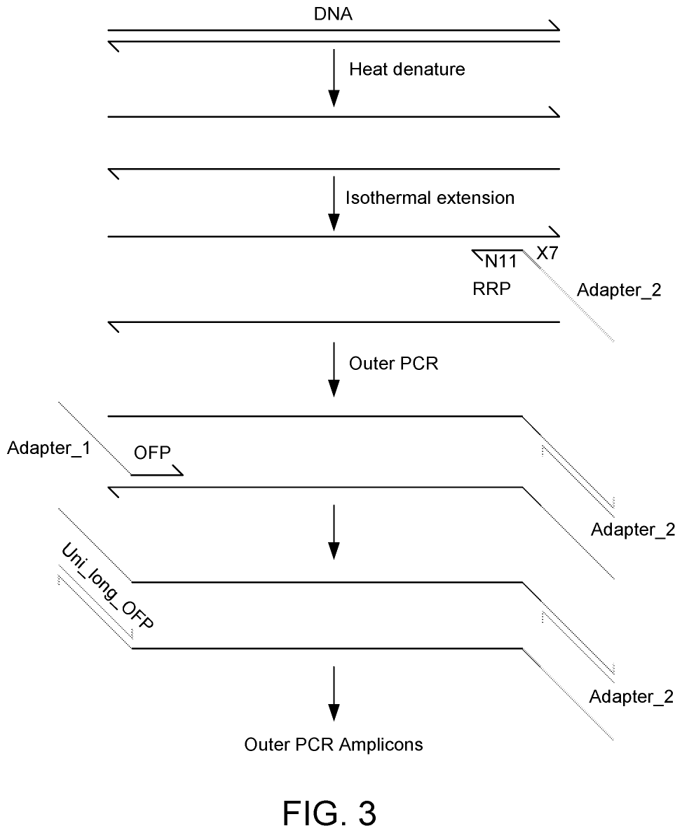

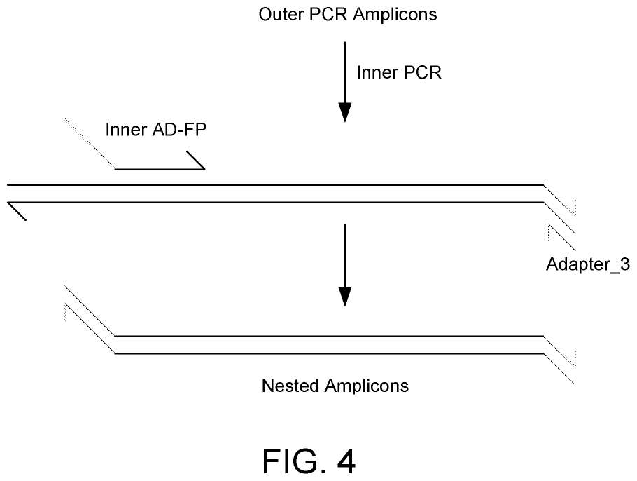

Generally, and without being limiting, the RRP approaches use an outer polymerase extension step followed by an inner PCR step. The outer polymerase extension step uses a gene-specific Outer Forward Primer and RRP primers. See FIGS. 2 and 3. The inner PCR step makes use of Inner Forward Primers. See FIG. 4.

In one aspect, this disclosure provides a method of preparing a sequencing library, the method comprising: (a) introducing to a sample comprising at least one nucleic acid template molecule: (i) at least one Randomer Reverse Primer (RRP), where the at least one RRP comprises a Randomer Sequence (RS) positioned within the 3′ region of the at least one RRP and a first universal adapter sequence positioned 5′ to the RS: (ii) a first polymerase; and (iii) a reaction buffer to create a first mixture: (b) subjecting the first mixture to a first polymerase extension step to generate a second mixture comprising at least one extended product: (c) introducing to the second mixture: (i) at least one Inner

Forward Primer (IFP) that binds to an IFP binding site on the at least one nucleic acid template molecule: (ii) a second adapter sequence: (iii) a thermostable polymerase; and (iv) reagents for thermostable polymerase activity to generate a third mixture; and (d) subjecting the third mixture to a second polymerase extension step to generate at least one amplicon of the at least one nucleic acid template molecule. In an aspect, the method further comprises introducing to the sample at least one Outer Forward Primer (OFP) in step (a).

In one aspect, this disclosure provides a method of preparing a sequencing library, the method comprising: (a) introducing to a sample comprising at least one nucleic acid template molecule: (i) at least one Randomer Reverse Primer (RRP), where the at least one RRP comprises a Randomer Sequence (RS) positioned on the 3′ region of the at least one RRP and a first universal adapter sequence positioned 5′ to the RS: (ii) at least one Outer Forward Primer (OFP): (iii) a first polymerase; and (iv) a reaction buffer to create a first mixture: (b) subjecting the first mixture to a first polymerase extension step to generate a second mixture comprising at least one extended product: (c) introducing to the second mixture: (i) at least one Inner Forward Primer (IFP) that binds to an IFP binding site on the at least one nucleic acid template molecule: (ii) a second adapter sequence: (iii) a thermostable polymerase, and (iv) reagents for thermostable polymerase activity to generate a third mixture; and (d) subjecting the third mixture to a second polymerase extension step to generate at least one amplicon of the at least one nucleic acid template molecule.

As used herein, a “sequencing library” refers to a pool of DNA fragments comprising at least one adapter. Adapters are typically designed to interact with a specific sequencing platform/instrument. Any sequencing method that can sequence a sequencing library is suitable for the methods provided herein. Non-limiting examples of sequencing methods include Sanger sequencing, and methods of sequencing that use an instrument selected from the group consisting of an Oxford Nanopore sequencer, a PacBio sequencer, an Illumina Miseq sequencer, an Illumina MiniSeq sequencer, an Illumina NextSeq sequencer, an Ion Torrent sequencer, and an Illumina Hiseq sequencer.

As used herein, a “nucleic acid template molecule” refers to a nucleic acid molecule that comprises a sequence that is desired to be amplified. In an aspect, a nucleic acid template molecule is a DNA molecule. In an aspect, a nucleic acid template molecule is an RNA molecule. In an aspect, a template nucleic acid molecule is a complementary DNA (cDNA) molecule. In an aspect, a nucleic acid template molecule is a single-stranded nucleic acid molecule. In an aspect, a nucleic acid template molecule is a double-stranded nucleic acid template molecule.

A nucleic acid template molecule can be from any organism. In an aspect, a nucleic acid template molecule is a prokaryotic nucleic acid template molecule. In an aspect, a nucleic acid template molecule is a eukaryotic nucleic acid template molecule. In an aspect, a nucleic acid template molecule is a viral DNA template molecule. In an aspect, a nucleic acid template molecule is a plant DNA template molecule. In an aspect, a nucleic acid template molecule is a fungal DNA template molecule. In an aspect, a nucleic acid template molecule is a protozoan DNA template molecule. In an aspect, a nucleic acid template molecule is an animal DNA template molecule. In an aspect, a nucleic acid template molecule is a mammalian DNA template molecule. In an aspect, a nucleic acid template molecule is a primate DNA template molecule. In an aspect, a nucleic acid template molecule is a human DNA template molecule. In an aspect, a nucleic acid template molecule is a human cancer cell DNA template molecule. In an aspect, a human cancer cell is selected from the group consisting of a lung cancer cell, a breast cancer cell, a prostate cancer cell, an ovarian cancer cell, colorectal cancer cell, a gastric cancer cell, and an endometrial cancer cell.

As used herein, a “cell line” refers to a culture of animal cells that is propagated repeatedly, and sometimes indefinitely, in an in vitro system. A cell line can be from any animal, including, without being limiting, a mammal, a fish, a bird, a reptile, and an amphibian. In an aspect, a cell line is a human cell line. In an aspect, a cell line is a human cancer cell line. Nucleic acid template molecules can be isolated from a cell line.

As used herein, a “clinical patient sample” refers to a sample obtained from a cell, blood, plasma, tissue, organ, or combination thereof from a patient, where the same comprises at least one nucleic acid template molecule. In an aspect, a patient is a human. In an aspect, a patient is a non-human animal. In an aspect, a patient is a mammal. In an aspect, a patient is selected from the group consisting of a mouse, a rat, a cat, a dog, a monkey, a chimpanzee, a cow, and a horse. In an aspect, a clinical patient sample is obtained from a tissue selected from the group consisting of epithelial tissue, connective tissue, muscle tissue, and nervous tissue. In an aspect, a clinical patient sample is obtained from a cell of an organ system selected from the group consisting of the respiratory system, the digestive or excretory system, the circulatory system, the urinary system, the integumentary system, the skeletal system, the muscular system, the endocrine system, the lymphatic system, the nervous system, and the reproductive system. Nucleic acid molecules can be isolated from a clinical patient sample.

In an aspect, a template nucleic acid molecule comprises an exon. In an aspect, a template nucleic acid molecule comprises an intron. In an aspect, a template nucleic acid molecule comprises at least part of one exon. In an aspect, a template nucleic acid molecule comprises at least part of one intron. In an aspect, a template nucleic acid molecule comprises at least part of one exon, at least part of one intron, or both. In an aspect, a template nucleic acid molecule comprises at least one gene. In an aspect, a template nucleic acid molecule comprises a plurality of genes. In an aspect, a template nucleic acid molecule comprises at least one gene fusion. In an aspect, a template nucleic acid molecule comprises a plurality of gene fusions. In an aspect, a template nucleic acid molecule comprises a non-coding nucleic acid molecule.

As used herein, an “exon” refers to a part of a gene that encodes a part of a final mature RNA after splicing. Exons encode amino acids during protein translation.

As used herein, an “intron” refers to a part of a gene that is spliced out of an RNA transcript prior to protein translation.

In an aspect, a template nucleic acid molecule comprises at least one long region of interest comprising a length of at least 50 nucleotides. In an aspect, a template nucleic acid molecule comprises at least one long region of interest comprising a length of at least 100 nucleotides. In an aspect, a template nucleic acid molecule comprises at least one long region of interest comprising a length of at least 250 nucleotides. In an aspect, a template nucleic acid molecule comprises at least one long region of interest comprising a length of at least 500 nucleotides. In an aspect, a template nucleic acid molecule comprises at least one long region of interest comprising a length of at least 1000 nucleotides.

As used herein, a “Randomer Reverse Primer” (RRP) refers to a primer comprising a random sequence that is suitable for use in polymerase extension. In an aspect, an RRP is a DNA molecule. In an aspect, a method comprises at least one RRP. In an aspect, a method comprises a plurality of RRPs. In an aspect, a method comprises at least 10 RRPs. In an aspect, a method comprises at least 50 RRPs. In an aspect, a method comprises at least 100 RRPs. In an aspect, a method comprises at least 500 RRPs. In an 30 aspect, a method comprises at least 1000 RRPs. A non-limiting example of an RRP is provided as SEQ ID NO: 1.

In an aspect, an RRP comprises a Randomer Sequence (RS) and a universal adapter sequence. In an aspect, the RS is positioned within the 3′ region of the RRP, and the universal adapter sequence is positioned 5′ to the RS. In an aspect, a universal adapter sequence is positioned at the 5′ end of an RRP. In an aspect, an RRP is positioned at the 3′end of an RRP.

A “Randomer Sequence” comprises a random nucleotide sequence. In an aspect, an RS comprises at least one degenerate nucleotide selected from the group consisting of R, Y, S, W, K, M, B, D, H, V, and N. In an aspect, an RS comprises at least two degenerate nucleotides selected from the group consisting of R, Y, S, W, K, M, B, D, H, V, and N. In an aspect, an RS comprises at least three degenerate nucleotides selected from the group consisting of R, Y, S, W, K, M, B, D, H, V, and N. In an aspect, an RS comprises at four degenerate nucleotides selected from the group consisting of R, Y, S, W, K, M, B, D, H, V, and N. In an aspect, an RS comprises at least five degenerate nucleotides selected from the group consisting of R, Y, S, W, K, M, B, D, H, V, and N. In an aspect, at least 5% of the nucleotides of an RS comprise degenerate nucleotides selected from the group consisting of R, Y, S, W, K, M, B, D, H, V, and N. In an aspect, at least 10% of the nucleotides of an RS comprise degenerate nucleotides selected from the group consisting of R, Y, S, W, K, M, B, D, H, V, and N. In an aspect, at least 15% of the nucleotides of an RS comprise degenerate nucleotides selected from the group consisting of R, Y, S, W, K, M, B, D, H, V, and N. In an aspect, at least 20% of the nucleotides of an RS comprise degenerate nucleotides selected from the group consisting of R, Y, S, W, K, M, B, D, H, V, and N. In an aspect, at least 25% of the nucleotides of an RS comprise degenerate nucleotides selected from the group consisting of R, Y, S, W, K, M, B, D, H, V, and N.

Regarding nucleotides, A refers to adenine, T refers to thymine, G refers to guanine, C refers to cytosine, R refers to an adenine or guanine: Y refers to a cytosine or thymine: S refers to a cytosine or guanine: W refers to an adenine or thymine: K refers to a guanine or thymine: M refers to an adenine or cytosine: B refers to a cytosine, guanine, 25 or thymine: D refers to an adenine, guanine, or thymine: H refers to an adenine, cytosine, or thymine: V refers to an adenine, cytosine, or guanine; and N refers to an adenine, cytosine, guanine, or thymine.

In an aspect, an RS comprises at least 3 nucleotides. In an aspect, an RS comprises at least 3 nucleotides. In an aspect, an RS comprises at least 4 nucleotides. In an aspect, an RS comprises at least 5 nucleotides. In an aspect, an RS comprises at least 6 nucleotides. In an aspect, an RS comprises at least 7 nucleotides. In an aspect, an RS comprises at least 8 nucleotides. In an aspect, an RS comprises at least 9 nucleotides. In an aspect, an RS comprises at least 10 nucleotides. In an aspect, an RS comprises at least 11 nucleotides. In an aspect, an RS comprises at least 12 nucleotides. In an aspect, an RS comprises at least 13 nucleotides. In an aspect, an RS comprises at least 14 nucleotides. In an aspect, an RS comprises at least 15 nucleotides. In an aspect, an RS comprises at least 20 nucleotides. In an aspect, an RS comprises at least 25 nucleotides. In an aspect, an RS comprises at least 30 nucleotides. In an aspect, an RS comprises at least 40 nucleotides. In an aspect, an RS comprises between 3 nucleotides and 50 nucleotides. In an aspect, an RS comprises between 3 nucleotides and 40 nucleotides. In an aspect, an RS comprises between 3 nucleotides and 30 nucleotides. In an aspect, an RS comprises between 3 nucleotides and 20 nucleotides. In an aspect, an RS comprises between 10 nucleotides and 50 nucleotides. In an aspect, an RS comprises between 10 nucleotides and 40 nucleotides. In an aspect, an RS comprises between 10 nucleotides and 30 nucleotides. In an aspect, an RS comprises between 20 nucleotides and 30 nucleotides.

In an aspect, a universal adapter sequence is an Adapter_1 sequence. An Adapter_1 sequence is linked to a GSOFP. A non-limiting example of an Adapter_1 sequence is provided as SEQ ID NO: 4. In an aspect, a universal adapter sequence is an Adapter_2 sequence. An Adapter_2 sequence is linked to an RRP. A non-limiting example of an Adapter 2 sequence is provided as SEQ ID NO: 2. In an aspect, a universal adapter sequence is an Adapter_3 sequence. An Adapter_3 sequence is part of an Adapter_2 sequence. A non-limiting example of an Adapter_3 sequence is SEQ ID NO: 3.

In an aspect, an adapter sequence is a universal adapter sequence. In an aspect, an adapter sequence is a sequencing adapter sequence.

In an aspect, an adapter sequence is an Adapter_1 sequence. In an aspect, an adapter sequence is an Adapter_2 sequence. In an aspect, an adapter sequence is an Adapter_3 sequence.

In an aspect, an RRP further comprises a Non-Relevant Sequence (NRS). As used herein, a “Non-Relevant Sequence” refers to nucleotides of an RRP that are not part of an RS, a universal adapter sequence, or another defined region (e.g. a UMI, MSS, or hairpin). In an aspect, a NRS does not hybridize directly with a nucleic acid template molecule. In an aspect, an NRS is positioned between an RS and a universal adapter sequence. In an aspect, an NRS is positioned 5′ to an RS and 3′ to an adapter sequence. In an aspect, an NRS is positioned 5′ to an RS and 3′ to a universal adapter sequence.

In an aspect, an NRS comprises less than 50% complementarity or identity with a nucleic acid template molecule. In an aspect, an NRS comprises less than 40% complementarity or identity with a nucleic acid template molecule. In an aspect, an NRS comprises less than 30% complementarity or identity with a nucleic acid template molecule. In an aspect, an NRS comprises less than 20% complementarity or identity with a nucleic acid template molecule. In an aspect, an NRS comprises less than 10% complementarity or identity with a nucleic acid template molecule. In an aspect, an NRS is not complementary or identical to a nucleic acid template molecule.

In an aspect, an NRS comprises less than 50% complementarity or identity with an adapter sequence. In an aspect, an NRS comprises less than 50% complementarity or identity with a universal adapter sequence. In an aspect, an NRS comprises less than 40% complementarity or identity with an adapter sequence. In an aspect, an NRS comprises less than 40% complementarity or identity with a universal adapter sequence. In an aspect, an NRS comprises less than 30% complementarity or identity with an adapter sequence. In an aspect, an NRS comprises less than 30% complementarity or identity with a universal adapter sequence. In an aspect, an NRS comprises less than 20% complementarity or identity with an adapter sequence. In an aspect, an NRS comprises less than 20% complementarity or identity with a universal adapter sequence. In an aspect, an NRS comprises less than 10% complementarity or identity with an adapter sequence. In an aspect, an NRS comprises less than 10% complementarity or identity with a universal adapter sequence. In an aspect, an NRS is not complementary or identical to an adapter sequence. In an aspect, an NRS is not complementary or identical to a universal adapter sequence.

In an aspect, an NRS comprises at least 3 nucleotides. In an aspect, an NRS comprises at least 4 nucleotides. In an aspect, an NRS comprises at least 5 nucleotides. In an aspect, an NRS comprises at least 6 nucleotides. In an aspect, an NRS comprises at least 7 nucleotides. In an aspect, an NRS comprises at least 8 nucleotides. In an aspect, an NRS comprises at least 9 nucleotides. In an aspect, an NRS comprises at least 10 nucleotides. In an aspect, an NRS comprises at least 12 nucleotides. In an aspect, an NRS comprises at least 15 nucleotides. In an aspect, an NRS comprises at least 18 nucleotides. In an aspect, an NRS comprises at least 20 nucleotides. In an aspect, an NRS comprises at least 25 nucleotides. In an aspect, an NRS comprises at least 30 nucleotides. In an aspect, an NRS comprises between 3 nucleotides and 30 nucleotides. In an aspect, an NRS comprises between 3 nucleotides and 25 nucleotides. In an aspect, an NRS comprises between 3 nucleotides and 20 nucleotides. In an aspect, an NRS comprises between 5 nucleotides and 30 nucleotides. In an aspect, an NRS comprises between 5 nucleotides and 25 nucleotides. In an aspect, an NRS comprises between 5 nucleotides and 20 nucleotides. In an aspect, an NRS comprises between 5 nucleotides and 15 nucleotides. In an aspect, an NRS comprises between 10 nucleotides and 20 nucleotides. In an aspect, an NRS comprises between 15 nucleotides and 20 nucleotides.

In an aspect, an RRP further comprises a Mixture of Specific Sequences (MSS). In an aspect, an MSS is positioned at the 3′ end of the RRP. In an aspect, an MSS comprises between 2 nucleotides and 5 nucleotides. In an aspect, an MSS comprises adenine (A), thymine (T), guanine (G) and/or cytosine (C) nucleotides. Inclusion of an MSS in an RRP helps avoid or reduce primer dimer formation.

In an aspect, an RRP further comprises a Unique Molecular Identifier (UMI) sequence. As used herein, a “Unique Molecular Identifier” refers to a unique nucleotide sequence that serves as a molecular barcode for an individual molecule. UMIs are often attached to DNA molecules in a sample library to uniquely tag each molecule. UMIs enable error correction and increased accuracy during sequencing of DNA molecules. In an aspect, a UMI sequence is positioned 5′ to an RS sequence. In an aspect, a UMI sequence is positioned 3′ to a universal adapter sequence. In aspect, a UMI sequence comprises at least one degenerate nucleotide selected from the group consisting of R, Y, S, W, K, M, B, D, H, V, and N. In an aspect, a UMI sequence comprises a mixture of between 10 and 100 defined DNA sequences with a minimum pairwise Hamming distance of between 2 and 5. In an aspect, a UMI sequence comprises a mixture of between 10 and 1000 defined DNA sequences with a minimum pairwise Levenschtein distance of between 2 and 5. In an aspect, a UMI comprises between 6 nucleotides and 20 nucleotides. In an aspect, a UMI comprises between 8 nucleotides and 20 nucleotides. In an aspect, a UMI comprises between 10 nucleotides and 20 nucleotides. In an aspect, a UMI comprises between 15 nucleotides and 20 nucleotides. In an aspect, a UMI comprises at least 6 nucleotides. In an aspect, a UMI comprises at least 8 nucleotides. In an aspect, a UMI comprises at least 10 nucleotides. In an aspect, a UMI comprises at least 12 nucleotides. In an aspect, a UMI comprises at least 15 nucleotides.

In an aspect, an RRP further comprises a Sample Barcode (SB) sequence. Sample Barcode sequences allow sequences to be identified and sorted when multiplex sequencing is undertaken. In an aspect, an SB sequence is positioned 5′ to an RS sequence. In an aspect, an SB sequence is positioned 3′ to a universal adapter sequence.

As used herein, a “5′ region” refers to anywhere in the 5′ half of a given nucleic acid molecule. The other half of the given nucleic acid molecule is then referred to as the “3′ region,” which refers to anywhere in the 3′ half of the nucleic acid molecule. As used herein, “upstream” refers to the 5′ side of a locus. As used herein, “downstream” refers to the 3′ side of a locus.

As used herein, a “positive strand” of DNA or cDNA refers to the sense strand of DNA. Typically, the positive strand of DNA corresponds to the sequence of an RNA transcript that is capable of being translated into a sequence of amino acids. In an aspect, a primer provided herein binds to a binding site on the positive strand of a DNA or cDNA molecule

As used herein, a “negative strand” of DNA or cDNA refers to the anti-sense strand of DNA. In an aspect, a primer provided herein binds to a binding site on the negative strand of a DNA or cDNA molecule.

As used herein, “polymerase” refers to an enzyme that synthesizes long chains of nucleic acids. As a non-limiting example, a DNA polymerase synthesizes DNA molecules, and an RNA polymerase synthesizes RNA molecules. Non-limiting examples of polymerases are categorized under by the International Union of Biochemistry and Molecular Biology under the Enzyme Catalog (EC) numbers EC 2.7.7.6, EC 2.7.7.7, EC 2.7.7.19, EC 2.7.7.48, and EC 2.7.7.49. In an aspect, a polymerase is a DNA polymerase. In an aspect, a polymerase is an RNA polymerase.

In an aspect, a polymerase is a thermostable polymerase. In an aspect, a thermostable polymerase is a thermostable DNA polymerase. In an aspect, a thermostable polymerase is a thermostable RNA polymerase. As used herein, a “thermostable polymerase” refers to a polymerase that is capable of withstanding temperatures of 70° C. and higher without denaturing or otherwise losing the ability to function. In an aspect, a thermostable polymerase is selected from the group consisting of Taq polymerase, Phusion® polymerase, Q5® polymerase, KAPA HiFi polymerase, Vent® DNA polymerase, LongAmp® Taq DNA polymerase, and OneTaq® DNA polymerase.

In an aspect, a polymerase is an isothermal polymerase. In an aspect, an isothermal polymerase is an isothermal DNA polymerase. In an aspect, an isothermal polymerase is an isothermal RNA polymerase. As used herein, an “isothermal polymerase” refers to a polymerase that is capable of functioning at (e.g., synthesize nucleic acid molecules given the proper reagents and starting materials), temperatures of 65° C. or lower. In an aspect, an isothermal polymerase is selected from the group consisting of a phi29 DNA polymerase, a Bst DNA polymerase, a Bst 2.0 DNA polymerase, a Bst 3.0 DNA polymerase, a T7 DNA polymerase, a T4 DNA polymerase, and a DNA polymerase 1 large (Klenow) fragment.

In an aspect, a polymerase is selected from the group consisting of phi29 DNA polymerase, DNA polymerase 1, large (Klenow) fragment, Klenow fragment, Bst DNA polymerase, T4 DNA polymerase, T7 DNA polymerase, Taq polymerase, Phusion R polymerase, Q5R: polymerase, KAPA HiFi polymerase, Vent® DNA polymerase, LongAmp® Taq DNA polymerase, and OneTaq® DNA polymerase. In an aspect, a polymerase is selected from the group consisting of Taq polymerase, Phusion R polymerase, Q5R; polymerase, KAPA HiFi polymerase, Vent® DNA polymerase, LongAmp® Taq DNA polymerase, and OneTaq® DNA polymerase. In an aspect, a polymerase is selected from the group consisting of a phi29 DNA polymerase, a Bst DNA polymerase, a Bst 2.0 DNA polymerase, a Bst 3.0 DNA polymerase, a T7 DNA polymerase, a T4 DNA polymerase, and a DNA polymerase 1 large (Klenow) fragment.

In an aspect, a mixture comprises one or more reagents necessary for polymerase activity. In an aspect, a mixture comprises one or more reagents necessary for thermostable polymerase activity. Non-limiting examples of reagents necessary for polymerase activity include dNTPs, buffers, magnesium, phosphatase, betaine, dimethyl sulfoxide, and tetramethylammonium chloride.

As used herein, “reaction buffer” refers to a buffer that provides a suitable chemical environment for polymerase activity. In an aspect, a reaction buffer comprises a pH between 8.0 and 9.5. In an aspect, a reaction buffer comprises Tris-HCl. In an aspect, a buffer comprises KCl.

As used herein, a “polymerase extension step” refers to subjecting a mixture comprising a polymerase to conditions suitable for allowing the polymerase to extend one or more primers and create at least one extended product. In an aspect, a polymerase extension step comprises a duration of between 10 seconds and 6 hours. In an aspect, a polymerase extension step comprises a duration of between 10 seconds and 4 hours. In an aspect, a polymerase extension step comprises a duration of between 10 seconds and 3 hours. In an aspect, a polymerase extension step comprises a duration of between 10 seconds and 2 hours. In an aspect, a polymerase extension step comprises a duration of between 10 seconds and 1 hour. In an aspect, a polymerase extension step comprises a duration of between 10 seconds and 30 minutes. In an aspect, a polymerase extension step comprises a duration of between 10 minutes and 6 hours. In an aspect, a polymerase extension step comprises a duration of between 10 minutes and 4 hours. In an aspect, a polymerase extension step comprises a duration of between 10 minutes and 2 hours. In an aspect, a polymerase extension step comprises a duration of at least 10 seconds. In an aspect, a polymerase extension step comprises a duration of at least 30 seconds. In an aspect, a polymerase extension step comprises a duration of at least 1 minute. In an aspect, a polymerase extension step comprises a duration of at least 2 minutes. In an aspect, a polymerase extension step comprises a duration of at least 5 minutes. In an aspect, a polymerase extension step comprises a duration of at least 10 minutes. In an aspect, a polymerase extension step comprises a duration of at least 30 minutes. In an aspect, a polymerase extension step comprises a duration of at least 45 minutes. In an aspect, a polymerase extension step comprises a duration of at least 1 hour. In an aspect, a polymerase extension step comprises a duration of at least 2 hours. In an aspect, a polymerase extension step comprises a duration of at least 3 hours. In an aspect, a polymerase extension step comprises a duration of at least 4 hours. In an aspect, a polymerase extension step comprises a duration of at least 5 hours.

In an aspect, a polymerase extension step comprises isothermal extension. In an aspect, a first polymerase extension step comprises isothermal extension. As used herein, “isothermal extension” refers to extension of a primer by a polymerase without heat denaturing a template nucleic acid molecule. In an aspect, an isothermal extension does not comprise thermal cycling. In an aspect, an isothermal extension occurs at a temperature between 20° C., and 65° C. In an aspect, an isothermal extension occurs at a temperature between 20° C., and 60° C. In an aspect, an isothermal extension occurs at a temperature between 20° C., and 50° C. In an aspect, an isothermal extension occurs at a temperature between 20° C., and 40° C. In an aspect, an isothermal extension occurs at a temperature between 30° C., and 65° C. In an aspect, an isothermal extension occurs at a temperature between 40° C., and 65° C. In an aspect, an isothermal extension occurs at a temperature between 50° C., and 65° C. In an aspect, an isothermal extension occurs at a temperature between 60° C., and 65° C. In an aspect, an isothermal extension occurs at a temperature between 30° C., and 60° C. In an aspect, an isothermal extension occurs at a temperature between 30° C., and 50° C.

In an aspect, a polymerase extension step comprises thermal cycling. In an aspect, a first polymerase extension step comprises thermal cycling. As used herein, “thermal cycling” refers to a controlled set of timed temperature changes. One “cycle” of thermal cycling comprises at least two stages. The first stage of a cycle comprises a first temperature maintained for a desired amount of time, and the second stage of a cycle comprises a second temperature maintained for a desired amount of time. In an aspect, a cycle further comprises a third stage comprising a third temperature maintained for a desired amount of time. In an aspect, a cycle further comprises a fourth stage comprising a fourth temperature maintained for a desired amount of time. Often, thermal cycling comprises repeating the same cycle several times.

In an aspect, a first, second, third, or fourth stage of a cycle comprises a temperature of less than 60° C. In an aspect, a first, second, third, or fourth stage of a cycle comprises a temperature of less than 70° C. In an aspect, a first, second, third, or fourth stage of a cycle comprises a temperature of less than 75° C. In an aspect, a first, second, third, or fourth stage of a cycle comprises a temperature of less than 80° C. In an aspect, a first, second, third, or fourth stage of a cycle comprises a temperature of less than 90° C. In an aspect, a first, second, third, or fourth stage of a cycle comprises a temperature of greater than 60° C. In an aspect, a first, second, third, or fourth stage of a cycle comprises a temperature of greater than 70° C. In an aspect, a first, second, third, or fourth stage of a cycle comprises a temperature of greater than 75° C. In an aspect, a first, second, third, or fourth stage of a cycle comprises a temperature of greater than 80° C. In an aspect, a first, second, third, or fourth stage of a cycle comprises a temperature of greater than 90° C.

In an aspect, a stage of a cycle comprises an annealing temperature of between 42° C., and 72° C. In an aspect, a stage of a cycle comprises an annealing temperature of between 45° C., and 72° C. In an aspect, a stage of a cycle comprises an annealing temperature of between 50° C., and 72° C. In an aspect, a stage of a cycle comprises an annealing temperature of between 55° C., and 72° C. In an aspect, a stage of a cycle comprises an annealing temperature of between 60° C., and 72° C. In an aspect, a stage of a cycle comprises an annealing temperature of between 65° C., and 72° C. In an aspect, a stage of a cycle comprises an annealing temperature of between 70° C., and 72° C.

As used herein, an “annealing temperature” is the temperature that is low enough to allow primers are able to bind to a nucleic acid template, but high enough that the formation of undesired, non-specific duplexes, or intramolecular hairpins amongst the primers is not favored. Those in the art routinely calculate appropriate annealing temperatures for a given primer sequence.

In an aspect, a first, second, third, or fourth stage of a cycle lasts for at least 1 second. In an aspect, a first, second, third, or fourth stage of a cycle lasts for at least 10 seconds. In an aspect, a first, second, third, or fourth stage of a cycle lasts for at least 30 seconds. In an aspect, a first, second, third, or fourth stage of a cycle lasts for at least 1 minute. In an aspect, a first, second, third, or fourth stage of a cycle lasts for at least 2 minutes. In an aspect, a first, second, third, or fourth stage of a cycle lasts for at least 10 minutes. In an aspect, a first, second, third, or fourth stage of a cycle lasts for at least 15 minutes. In an aspect, a first, second, third, or fourth stage of a cycle lasts for at least 30 minutes. In an aspect, a first, second, third, or fourth stage of a cycle lasts for at least 1 hour. In an aspect, a first, second, third, or fourth stage of a cycle lasts for at least 2 hours.

In an aspect, a first, second, third, or fourth stage of a cycle lasts for between 1 second and 3 hours. In an aspect, a first, second, third, or fourth stage of a cycle lasts for between 1 second and 2 hours. In an aspect, a first, second, third, or fourth stage of a cycle lasts for between 1 second and 1 hour. In an aspect, a first, second, third, or fourth stage of a cycle lasts for between 1 second and 30 minutes. In an aspect, a first, second, third, or fourth stage of a cycle lasts for between 1 second and 20 minutes. In an aspect, a first, second, third, or fourth stage of a cycle lasts for between 1 second and 15 minutes. In an aspect, a first, second, third, or fourth stage of a cycle lasts for between 1 second and 10 minutes. In an aspect, a first, second, third, or fourth stage of a cycle lasts for between 1 second and 5 minutes. In an aspect, a first, second, third, or fourth stage of a cycle lasts for between 1 second and 2 minutes. In an aspect, a first, second, third, or fourth stage of a cycle lasts for between 1 second and 1 minute. In an aspect, a first, second, third, or fourth stage of a cycle lasts for between 1 second and 30 seconds.

In an aspect, thermal cycling comprises at least 1 cycle. In an aspect, thermal cycling comprises at least 2 cycles. In an aspect, thermal cycling comprises at least 3 cycles. In an aspect, thermal cycling comprises at least 4 cycles. In an aspect, thermal cycling comprises at least 5 cycles. In an aspect, thermal cycling comprises at least 6 cycles. In an aspect, thermal cycling comprises at least 7 cycles. In an aspect, thermal cycling comprises at least 8 cycles. In an aspect, thermal cycling comprises at least 9 cycles. In an aspect, thermal cycling comprises at least 10 cycles. In an aspect, thermal cycling comprises at least 15 cycles. In an aspect, thermal cycling comprises at least 20 cycles. In an aspect, thermal cycling comprises at least 25 cycles. In an aspect, thermal cycling comprises at least 30 cycles. In an aspect, thermal cycling comprises at least 40 cycles. In an aspect, thermal cycling comprises at least 50 cycles.

In an aspect, thermal cycling comprises between 1 cycle and 60 cycles. In an aspect, thermal cycling comprises between 1 cycle and 50 cycles. In an aspect, thermal cycling comprises between 1 cycle and 40 cycles. In an aspect, thermal cycling comprises between 1 cycle and 30 cycles. In an aspect, thermal cycling comprises between 1 cycle and 20 cycles. In an aspect, thermal cycling comprises between 1 cycle and 10 cycles. In an aspect, thermal cycling comprises between 1 cycle and 5 cycles. In an aspect, thermal cycling comprises between 2 cycles and 60 cycles. In an aspect, thermal cycling comprises between 2 cycles and 40 cycles. In an aspect, thermal cycling comprises between 2 cycles and 20 cycles. In an aspect, thermal cycling comprises between 2 cycles and 10 cycles. In an aspect, thermal cycling comprises between 2 cycles and 8 cycles. In an aspect, thermal cycling comprises between 20 cycles and 60 cycles. In an aspect, thermal cycling comprises between 20 cycles and 40 cycles.

In an aspect, each cycle of thermal cycling comprises (a) a first stage comprising a temperature of at least 75° C. for between one second and one hour; and (b) a second stage comprising a temperature of less than 75° C. for between one second and two hours. In an aspect, each cycle of thermal cycling comprises (a) a first stage comprising a temperature of at least 80° C. for between one second and one hour; and (b) a second stage comprising a temperature of less than 80° C. for between one second and two hours. In an aspect, each cycle of thermal cycling comprises (a) a first stage comprising a temperature of at least 90° C. for between one second and one hour; and (b) a second stage comprising a temperature of less than 90° C. for between one second and two hours.

In an aspect, thermal cycling further comprises the use of at least one wildtype-specific blocker. In an aspect, thermal cycling further comprises the use of a plurality of wildtype-specific blockers. As used herein, a “wildtype-specific blocker” refers to an oligonucleotide comprising at least one continuous strand of from about 12 to about 100 nucleotides in length which strand preferably anneals to a to-be-blocked allele sequence (the wildtype sequence) relative to a non-blocked allele sequence. In an aspect, a wildtype-specific blocker further comprises a functional group or a nucleotide sequence at its 3′ end that prevents enzymatic extension during an amplification process such as polymerase chain reaction. In an aspect, a wildtype-specific blocker comprises a terminator to prevent 3′ to 5′ DNA polymerase exonuclease activity. In an aspect, a terminator is selected from the group consisting of a three-carbon (C3) spacer and DXXDM, where D is a match between the blocker sequence and the template nucleic acid molecule sequence, X is a mismatch between the blocker sequence and the template nucleic acid molecule sequence, and M is a C3 spacer. Additional terminators known in the art are also suitable for use. A non-limiting example of an additional terminator is a dideoxynucleotide.

In an aspect, at least one wildtype-specific blocker corresponds to a wildtype sequence that overlaps with at least one IFP by between 2 nucleotides and 30 nucleotides. In an aspect, at least one wildtype-specific blocker corresponds to a wildtype sequence that overlaps with at least one IFP by between 2 nucleotides and 25 nucleotides. In an aspect, at least one wildtype-specific blocker corresponds to a wildtype sequence that overlaps with at least one IFP by between 2 nucleotides and 20 nucleotides. In an aspect, at least one wildtype-specific blocker corresponds to a wildtype sequence that overlaps with at least one IFP by between 2 nucleotides and 15 nucleotides. In an aspect, at least one wildtype-specific blocker corresponds to a wildtype sequence that overlaps with at least one IFP by between 2 nucleotides and 10 nucleotides. In an aspect, at least one wildtype-specific blocker corresponds to a wildtype sequence that overlaps with at least one IFP by between 2 nucleotides and 5 nucleotides. In an aspect, at least one wildtype-specific blocker corresponds to a wildtype sequence that overlaps with at least one IFP by between 10 nucleotides and 30 nucleotides.

In an aspect, an overlap between a wildtype-specific blocker and an IFP comprises a standard free energy of binding between −2 kcal/mol and −4 kcal/mol. In an aspect, the sequence of an IFP that does not overlap with a wildtype-specific blocker comprises a standard free energy between −5 kcal/mol and −9 kcal/mol. In an aspect, the sequence of a wildtype-specific blocker that does not overlap with an IFP sequence comprises a standard free energy between −7 kcal/mol and −12 kcal/mol. Unless described otherwise, the standard free energy of binding is calculated based on an annealing temperature of 60° C., double-stranded DNA, and a Na⁺ concentration of 0.18 M.

In an aspect, an IFP, OFP, or an MFP comprises a standard free energy of between −11.5 kcal/mol and −12.5 kcal/mol in a standard PCR buffer.

As used herein, an “extended product” refers to a nucleic acid molecule produced by a polymerase. As a non-limiting example, an amplicon is an extended product. In an aspect, a polymerase extension step generates a mixture comprising at least one extended product. In an aspect, a polymerase extension step generates a mixture comprising a plurality of extended products.

In an aspect, a mixture comprising at least one extended product is diluted prior to adding additional components (e.g., primers, polymerases, reagents) to the mixture. Any suitable liquid can be used as a dilutant, including, without being limiting, water and buffers. In an aspect, a mixture is diluted at a ratio of at least 1:2. In an aspect, a mixture is diluted at a ratio of at least 1:4. In an aspect, a mixture is diluted at a ratio of at least 1:5. In an aspect, a mixture is diluted at a ratio of at least 1:10. In an aspect, a mixture is diluted at a ratio of at least 1:25. In an aspect, a mixture is diluted at a ratio of at least 1:50. In an aspect, a mixture is diluted at a ratio of at least 1:100. In an aspect, a mixture is diluted at a ratio of at least 1:250. In an aspect, a mixture is diluted at a ratio of at least 1:500. In an aspect, a mixture is diluted at a ratio of at least 1:1000. In an aspect, a mixture is diluted at a ratio of at least 1:2500. In an aspect, a mixture is diluted at a ratio of at least 1:5000. In an aspect, a mixture is diluted at a ratio of at least 1:10,000. In an aspect, a mixture is diluted at a ratio of at least 1:12,500. In an aspect, a mixture is diluted at a ratio between 1:10 and 1:10,000. In an aspect, a mixture is diluted at a ratio between 1:10 and 1:5000. In an aspect, a mixture is diluted at a ratio between 1:10 and 1:1000. In an aspect, a mixture is diluted at a ratio between 1:10 and 1:500. In an aspect, a mixture is diluted at a ratio between 1:10 and 1:100. In an aspect, a mixture is diluted at a ratio between 1:2 and 1:10,000.

In an aspect, at least one extended product, or a plurality of extended products, is purified from a mixture prior to adding additional components (e.g., primers, polymerases, reagents) to the at least one extended product or plurality of extended products. Purification of extended products can be performed using any suitable method known in the art. In an aspect, at least one extended product, or a plurality of extended products, is purified from a mixture using column purification. In an aspect, at least one extended product, or a plurality of extended products, is purified from a mixture using beads purification.

In an aspect, at least one amplicon, or a plurality of amplicons, is purified from a mixture. Purification of amplicons can be performed using any suitable method known in the art. In an aspect, at least one amplicon, or a plurality of amplicons, is purified from a mixture using column purification. In an aspect, at least one amplicon, or a plurality of amplicons, is purified from a mixture using beads purification.

In an aspect, an amplicon, or a plurality of amplicons, is diluted. Any suitable liquid can be used as a dilutant, including, without being limiting, water and buffers. In an aspect, an amplicon is diluted at a ratio of at least 1:2. In an aspect, an amplicon is diluted at a ratio of at least 1:4. In an aspect, an amplicon is diluted at a ratio of at least 1:5. In an aspect, an amplicon is diluted at a ratio of at least 1:10. In an aspect, an amplicon is diluted at a ratio of at least 1:25. In an aspect, an amplicon is diluted at a ratio of at least 1:50. In an aspect, an amplicon is diluted at a ratio of at least 1:100. In an aspect, an amplicon is diluted at a ratio of at least 1:250. In an aspect, an amplicon is diluted at a ratio of at least 1:500. In an aspect, an amplicon is diluted at a ratio of at least 1:1000. In an aspect, an amplicon is diluted at a ratio of at least 1:2500. In an aspect, an amplicon is diluted at a ratio of at least 1:5000. In an aspect, an amplicon is diluted at a ratio of at least 1:10,000. In an aspect, an amplicon is diluted at a ratio of at least 1:12,500. In an aspect, an amplicon is diluted at a ratio between 1:10 and 1:10,000. In an aspect, an amplicon is diluted at a ratio between 1:10 and 1:5000. In an aspect, an amplicon is diluted at a ratio between 1:10 and 1:1000. In an aspect, an amplicon is diluted at a ratio between 1:10 and 1:500. In an aspect, an amplicon is diluted at a ratio between 1:10 and 1:100. In an aspect, an amplicon is diluted at a ratio between 1:2 and 1:10,000.

As used herein, a “primer” refers to a chemically synthesized single-stranded oligonucleotide which is designed to anneal to a specific site on a template nucleic acid molecule. Without being limiting, a primer is used in a polymerase extension step to initiate DNA synthesis. In an aspect, a primer is a DNA molecule. In an aspect, a primer is an RNA molecule. In an aspect, a primer is a forward primer. In an aspect, a primer is a reverse primer. As used herein, a “forward primer” hybridizes to the anti-sense strand of dsDNA, and a “reverse primer” hybridizes to the sense strand of dsDNA. In an aspect, a forward primer comprises DNA. In an aspect, a reverse primer comprises DNA. In an aspect, a forward primer comprises RNA. In an aspect, a reverse primer comprises RNA.

In an aspect, a primer is an Inner Forward Primer (IFP). In an aspect, an IFP binds (e.g., hybridizes) to an IFP binding site on a template nucleic acid molecule. In an aspect, a primer is an Outer Forward Primer (OFP). In an aspect, an OFP binds (e.g., hybridizes) to an OFP binding site on a template nucleic acid molecule. In an aspect, a primer is a Middle Forward Primer (MFP). In an aspect, an MFP binds (e.g., hybridizes) to an MFP binding site on a template nucleic acid molecule. For a given gene or target sequence, an IFP binding site is positioned, at least partially, 3′ to an OFP binding site. For a given gene or target sequence, an MFP binding site partially overlaps an OFP binding site, an IFP binding site, or both. In an aspect, an IFP binding site, an OFP binding site, or an MFP binding site is on the positive strand of a DNA or cDNA molecule. In an aspect, an IFP binding site, an OFP binding site, or an MFP binding site is on the negative strand of a DNA or cDNA molecule.

In an aspect, a method provided herein comprises at least one IFP. In an aspect, a method provided herein comprises at least two IFPs. In an aspect, a method provided herein comprises at least five IFPs. In an aspect, a method provided herein comprises at least 10 IFPs. In an aspect, a method provided herein comprises at least 25 IFPs. In an aspect, a method provided herein comprises at least 50 IFPs. In an aspect, a method provided herein comprises a plurality of IFPs.

In an aspect, a method provided herein comprises at least one GSIFP. In an aspect, a method provided herein comprises at least two GSIFPs. In an aspect, a method provided herein comprises at least five GSIFPs. In an aspect, a method provided herein comprises at least 10 GSIFPs. In an aspect, a method provided herein comprises at least 25 GSIFPs. In an aspect, a method provided herein comprises at least 50 GSIFPs. In an aspect, a method provided herein comprises a plurality of GSIFPs.

In an aspect, a method provided herein comprises at least one OFP. In an aspect, a method provided herein comprises at least two OFPs. In an aspect, a method provided herein comprises at least five OFPs. In an aspect, a method provided herein comprises at least 10 OFPs. In an aspect, a method provided herein comprises at least 25 OFPs. In an aspect, a method provided herein comprises at least 50 OFPs. In an aspect, a method provided herein comprises a plurality of OFPs.

In an aspect, a method provided herein comprises at least one GSOFP. In an aspect, a method provided herein comprises at least two GSOFPs. In an aspect, a method provided herein comprises at least five GSOFPs. In an aspect, a method provided herein comprises at least 10 GSOFPs. In an aspect, a method provided herein comprises at least 25 GSOFPs. In an aspect, a method provided herein comprises at least 50 GSOFPs. In an aspect, a method provided herein comprises a plurality of GSOFPs.

In an aspect, a method provided herein comprises at least one MFP. In an aspect, a method provided herein comprises at least two MFPs. In an aspect, a method provided herein comprises at least five MFPs. In an aspect, a method provided herein comprises at least 10 MFPs. In an aspect, a method provided herein comprises at least 25 MFPs. In an aspect, a method provided herein comprises at least 50 MFPs. In an aspect, a method provided herein comprises a plurality of MFPs.

In an aspect, an IFP binding site is positioned 5′ on a template nucleic acid molecule as compared to a GSOFP binding site. In an aspect, a GSIFP binding site is positioned 5′ on a template nucleic acid molecule as compared to a GSOFP binding site. In an aspect, an IFP binding site is positioned 5′ on a template nucleic acid molecule as compared to an OFP binding site.

In an aspect, an OFP comprises a Gene Specific Outer Forward Primer (GSOFP) and an adapter sequence. In an aspect, an OFP comprises a GSOFP and a universal adapter sequence. A GSOFP is capable of binding (e.g. hybridizing) to a specific sequence in a template nucleic acid molecule. The GSOFP binds to a GSOFP binding site on the template nucleic acid molecule.

In an aspect, an IFP is a Gene Specific Inner Forward Primer (GSIFP). A GSOFP is capable of binding (e.g. hybridizing) to a specific sequence in a template nucleic acid molecule. The GSIFP binds to a GSIFP binding site on the template nucleic acid molecule.

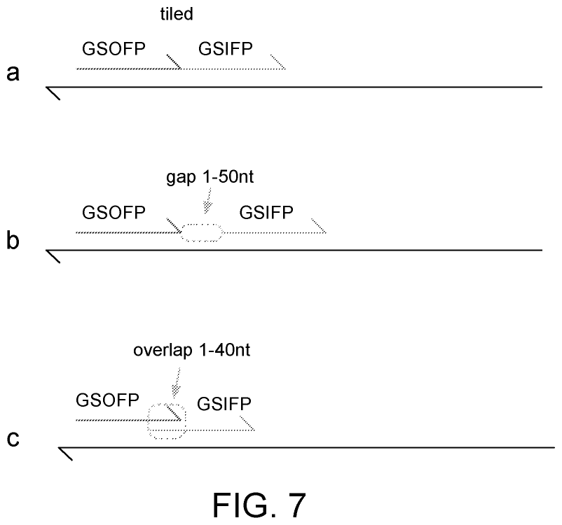

In an aspect, an OFP binding site and an IFP binding site overlap. In an aspect, a GSOFP binding site and a GSIFP binding site overlap. In an aspect, a GSOFP binding site and an IFP binding site overlap. In an aspect, an OFP binding site and a GSIFP binding site overlap. In an aspect, the 5′ end of an IFP binding site overlaps with the 3′ end of an OFP binding site. In an aspect, the 5′ end of an IFP binding site overlaps with the 3′ end of a GSOFP binding site. In an aspect, the 5′ end of a GSIFP binding site overlaps with the 3′ end of an OFP binding site. In an aspect, the 5′ end of a GSIFP binding site overlaps with the 3′ end of a GSOFP binding site. See FIG. 7c for an example of overlapping primer binding sites. In an aspect, an MFP binding site overlaps with an IFP binding site, an OFP binding site, or both. In an aspect, an MFP binding site overlaps with a GSIFP binding site, a GSOFP binding site, or both.

In an aspect, an overlap comprises at least 1 nucleotide. In an aspect, an overlap comprises at least 2 nucleotides. In an aspect, an overlap comprises at least 5 nucleotides. In an aspect, an overlap comprises at least 10 nucleotides. In an aspect, an overlap comprises at least 15 nucleotides. In an aspect, an overlap comprises at least 20 nucleotides. In an aspect, an overlap comprises at least 25 nucleotides. In an aspect, an overlap comprises at least 30 nucleotides. In an aspect, an overlap comprises at least 35 nucleotides. In an aspect, an overlap comprises at least 40 nucleotides. In an aspect, an overlap comprises at least 45 nucleotides. In an aspect, an overlap comprises at least 50 nucleotides. In an aspect, an overlap comprises between 1 nucleotide and 50 nucleotides. In an aspect, an overlap comprises at least 50 nucleotides. In an aspect, an overlap comprises between 1 nucleotide and 45 nucleotides. In an aspect, an overlap comprises at least 50 nucleotides. In an aspect, an overlap comprises between 1 nucleotide and 40 nucleotides. In an aspect, an overlap comprises at least 50 nucleotides. In an aspect, an overlap comprises between 1 nucleotide and 30 nucleotides. In an aspect, an overlap comprises at least 50 nucleotides. In an aspect, an overlap comprises between 1 nucleotide and 20 nucleotides. In an aspect, an overlap comprises at least 50 nucleotides. In an aspect, an overlap comprises between 10 nucleotides and 40 nucleotides. In an aspect, an overlap comprises between 5 nucleotides and 40 nucleotides.

In an aspect, an IFP binding site and an OFP binding site do not overlap. In an aspect, an IFP binding site and a GSOFP binding site do not overlap. In an aspect, a GSIFP binding site and an OFP binding site do not overlap. In an aspect, a GSIFP binding site and a GSOFP binding site do not overlap.

When two primer binding sites are positioned such that there are 0 nucleotides between them, and the two primer binding sites do not overlap, the primer binding sites are considered to be “adjacent.” See, FIG. 7a for an example of adjacent binding sites. In an aspect, an IFP binding site and an OFP binding site are adjacent. In an aspect, an IFP binding site and a GSOFP binding site are adjacent. In an aspect, a GSIFP binding site and an OFP binding site are adjacent. In an aspect, a GSIFP binding site and a GSOFP binding site are adjacent. When primers are designed adjacent to each other, and they cover the entire region of interest, the are said to “tile” the region of interest.

In an aspect, a plurality of IFPs is used to tile a long region of interest. In an aspect, a tile is conducted in two orientations, one of which is based on the positive strand of a template nucleic acid molecule, and the other of which is based on the negative strand of the template nucleic acid molecule.

When two primer binding sites do not overlap and are not adjacent, the are considered to have a “gap” between them. See FIG. 7b for an example of a gap. In an aspect, there is a gap between an IFP binding site and an OFP binding site. In an aspect, there is a gap between an IFP binding site and a GSOFP binding site. In an aspect, there is a gap between a GSIFP binding site and an OFP binding site. In an aspect, there is a gap between a GSIFP binding site and a GSOFP binding site. In an aspect, a gap is positioned between the 3′ end of an OFP binding site and the 5′ end of an IFP binding site. In an aspect, a gap is positioned between the 3′ end of an OFP binding site and the 5′ end of a GSIFP binding site. In an aspect, a gap is positioned between the 3′ end of a GSOFP binding site and the 5′ end of an IFP binding site. In an aspect, a gap is positioned between the 3′ end of a GSOFP binding site and the 5′ end of a GSIFP binding site.

In an aspect, a gap comprises at least 1 nucleotide. In an aspect, a gap comprises at least 2 nucleotides. In an aspect, a gap comprises at least 5 nucleotides. In an aspect, a gap comprises at least 10 nucleotides. In an aspect, a gap comprises at least 15 nucleotides. In an aspect, a gap comprises at least 20 nucleotides. In an aspect, a gap comprises at least 25 nucleotides. In an aspect, a gap comprises at least 30 nucleotides. In an aspect, a gap comprises at least 40 nucleotides. In an aspect, a gap comprises at least 50 nucleotides. In an aspect, a gap comprises at least 60 nucleotides. In an aspect, a gap comprises at least 75 nucleotides. In an aspect, a gap comprises between 1 nucleotide and 75 nucleotides. In an aspect, a gap comprises between 1 nucleotide and 60 nucleotides. In an aspect, a gap comprises between 1 nucleotide and 50 nucleotides. In an aspect, a gap comprises between 1 nucleotide and 40 nucleotides. In an aspect, a gap comprises between 1 nucleotide and 30 nucleotides. In an aspect, a gap comprises between 1 nucleotide and 20 nucleotides. In an aspect, a gap comprises between 1 nucleotide and 10 nucleotides.

In an aspect, an IFP, an OFP, or an MFP comprises at least 10 nucleotides. In an aspect, an IFP, an OFP, or an MFP comprises at least 15 nucleotides. In an aspect, an IFP, an OFP, or an MFP comprises at least 20 nucleotides. In an aspect, an IFP, an OFP, or an MFP comprises at least 25 nucleotides. In an aspect, an IFP, an OFP, or an MFP comprises at least 30 nucleotides. In an aspect, an IFP, an OFP, or an MFP comprises at least 35 nucleotides. In an aspect, an IFP, an OFP, or an MFP comprises at least 40 nucleotides. In an aspect, an IFP, an OFP, or an MFP comprises at least 45 nucleotides. In an aspect, an IFP, an OFP, or an MFP comprises at least 50 nucleotides. In an aspect, an IFP, an OFP, or an MFP comprises at least 75 nucleotides. In an aspect, an IFP, an OFP, or an MFP comprises at least 100 nucleotides. In an aspect, an IFP, an OFP, or an MFP comprises at between 10 nucleotides and 150 nucleotides. In an aspect, an IFP, an OFP, or an MFP comprises at between 10 nucleotides and 125 nucleotides. In an aspect, an IFP, an OFP, or an MFP comprises at between 10 nucleotides and 100 nucleotides. In an aspect, an IFP, an OFP, or an MFP comprises at between 10 nucleotides and 70 nucleotides. In an aspect, an IFP, an OFP, or an MFP comprises at between 10 nucleotides and 50 nucleotides. In an aspect, an IFP, an OFP, or an MFP comprises at between 10 nucleotides and 40 nucleotides. In an aspect, an IFP, an OFP, or an MFP comprises at between 20 nucleotides and 70 nucleotides. In an aspect, an IFP, an OFP, or an MFP comprises at between 25 nucleotides and 70 nucleotides. In an aspect, an IFP, an OFP, or an MFP comprises at between 20 nucleotides and 60 nucleotides. In an aspect, an IFP, an OFP, or an MFP comprises at between 20 nucleotides and 50 nucleotides.

In an aspect, a GSIFP or a GSOFP comprises at least 10 nucleotides. In an aspect, a GSIFP or a GSOFP comprises at least 15 nucleotides. In an aspect, a GSIFP or a GSOFP comprises at least 20 nucleotides. In an aspect, a GSIFP or a GSOFP comprises at least 25 nucleotides. In an aspect, a GSIFP or a GSOFP comprises at least 30 nucleotides. In an aspect, a GSIFP or a GSOFP comprises at least 35 nucleotides. In an aspect, a GSIFP or a GSOFP comprises at least 40 nucleotides. In an aspect, a GSIFP or a GSOFP comprises at least 45 nucleotides. In an aspect, a GSIFP or a GSOFP comprises at least 50 nucleotides. In an aspect, a GSIFP or a GSOFP comprises at least 75 nucleotides. In an aspect, a GSIFP or a GSOFP comprises at least 100 nucleotides. In an aspect, a GSIFP or a GSOFP comprises at between 10 nucleotides and 150 nucleotides. In an aspect, a GSIFP or a GSOFP comprises at between 10 nucleotides and 125 nucleotides. In an aspect, a GSIFP or a GSOFP comprises at between 10 nucleotides and 100 nucleotides. In an aspect, a GSIFP or a GSOFP comprises at between 10 nucleotides and 70 nucleotides. In an aspect, a GSIFP or a GSOFP comprises at between 10 nucleotides and 50 nucleotides. In an aspect, a GSIFP or a GSOFP comprises at between 10 nucleotides and 40 nucleotides. In an aspect, a GSIFP or a GSOFP comprises at between 20 nucleotides and 70 nucleotides. In an aspect, a GSIFP or a GSOFP comprises at between 25 nucleotides and 70 nucleotides. In an aspect, a GSIFP or a GSOFP comprises at between 20 nucleotides and 60 nucleotides. In an aspect, a GSIFP or a GSOFP comprises at between 20 nucleotides and 50 nucleotides.

In an aspect, an IFP, an OFP, or an MFP comprises a sequence that is at least 70% identical or complementary to the sequence of a template nucleic acid molecule. In an aspect, an IFP, an OFP, or an MFP comprises a sequence that is at least 75% identical or complementary to the sequence of a template nucleic acid molecule. In an aspect, an IFP, an OFP, or an MFP comprises a sequence that is at least 80% identical or complementary to the sequence of a template nucleic acid molecule. In an aspect, an IFP, an OFP, or an MFP comprises a sequence that is at least 85% identical or complementary to the sequence of a template nucleic acid molecule. In an aspect, an IFP, an OFP, or an MFP comprises a sequence that is at least 90% identical or complementary to the sequence of a template nucleic acid molecule. In an aspect, an IFP, an OFP, or an MFP comprises a sequence that is at least 95% identical or complementary to the sequence of a template nucleic acid molecule. In an aspect, an IFP, an OFP, or an MFP comprises a sequence that is at least 97.5% identical or complementary to the sequence of a template nucleic acid molecule. In an aspect, an IFP, an OFP, or an MFP comprises a sequence that is at least 99% identical or complementary to the sequence of a template nucleic acid molecule. In an aspect, an IFP, an OFP, or an MFP comprises a sequence that is 100% identical or complementary to the sequence of a template nucleic acid molecule.

In an aspect, a GSIFP or a GSOFP comprises a sequence that is at least 70% identical or complementary to the sequence of a template nucleic acid molecule. In an aspect, a GSIFP or a GSOFP comprises a sequence that is at least 75% identical or complementary to the sequence of a template nucleic acid molecule. In an aspect, a GSIFP or a GSOFP comprises a sequence that is at least 80% identical or complementary to the sequence of a template nucleic acid molecule. In an aspect, a GSIFP or a GSOFP comprises a sequence that is at least 85% identical or complementary to the sequence of a template nucleic acid molecule. In an aspect, a GSIFP or a GSOFP comprises a sequence that is at least 90% identical or complementary to the sequence of a template nucleic acid molecule. In an aspect, a GSIFP or a GSOFP comprises a sequence that is at least 95% identical or complementary to the sequence of a template nucleic acid molecule. In an aspect, a GSIFP or a GSOFP comprises a sequence that is at least 97.5% identical or complementary to the sequence of a template nucleic acid molecule. In an aspect, a GSIFP or a GSOFP comprises a sequence that is at least 99% identical or complementary to the sequence of a template nucleic acid molecule. In an aspect, a GSIFP or a GSOFP comprises a sequence that is 100% identical or complementary to the sequence of a template nucleic acid molecule.

In an aspect, an IFP or a plurality of IFPs is present at a concentration of at least 1 nM. In an aspect, an IFP or a plurality of IFPs is present at a concentration of at least 10 nM. In an aspect, an IFP or a plurality of IFPs is present at a concentration of at least 50 nM. In an aspect, an IFP or a plurality of IFPs is present at a concentration of at least 100 nM. In an aspect, an IFP or a plurality of IFPs is present at a concentration of at least 250 nM. In an aspect, an IFP or a plurality of IFPs is present at a concentration of at least 500 nM. In an aspect, an IFP or a plurality of IFPs is present at a concentration of at least 1000 nM. In an aspect, an IFP or a plurality of IFPs is present at a concentration of between 1 nM and 1000 nM. In an aspect, an IFP or a plurality of IFPs is present at a concentration of between 10 nM and 1000 nM. In an aspect, an IFP or a plurality of IFPs is present at a concentration of between 100 nM and 1000 nM. In an aspect, an IFP or a plurality of IFPs is present at a concentration of between 500 nM and 1000 nM. In an aspect, an IFP or a plurality of IFPs is present at a concentration of between 1 nM and 500 nM. In an aspect, an IFP or a plurality of IFPs is present at a concentration of between 1 nM and 100 nM. In an aspect, an IFP or a plurality of IFPs is present at a concentration of between 1 nM and 10 nM.

In an aspect, an OFP or a plurality of OFPs is present at a concentration of at least 1 nM. In an aspect, an OFP or a plurality of OFPs is present at a concentration of at least 10 nM. In an aspect, an OFP or a plurality of OFPs is present at a concentration of at least 50 nM. In an aspect, an OFP or a plurality of OFPs is present at a concentration of at least 100 nM. In an aspect, an OFP or a plurality of OFPs is present at a concentration of at least 250 nM. In an aspect, an OFP or a plurality of OFPs is present at a concentration of at least 500 nM. In an aspect, an OFP or a plurality of OFPs is present at a concentration of at least 1000 nM. In an aspect, an OFP or a plurality of OFPs is present at a concentration of between 1 nM and 1000 nM. In an aspect, an OFP or a plurality of OFPs is present at a concentration of between 10 nM and 1000 nM. In an aspect, an OFP or a plurality of OFPs is present at a concentration of between 100 nM and 1000 nM. In an aspect, an OFP or a plurality of OFPs is present at a concentration of between 500 nM and 1000 nM. In an aspect, an OFP or a plurality of OFPs is present at a concentration of between 1 nM and 500 nM. In an aspect, an OFP or a plurality of OFPs is present at a concentration of between 1 nM and 100 nM. In an aspect, an OFP or a plurality of OFPs is present at a concentration of between 1 nM and 10 nM.

In an aspect, the total concentration of all OFPs used in a mixture is less than 10 μM. In an aspect, the total concentration of all OFPs used in a mixture is less than 7.5 μM. In an aspect, the total concentration of all OFPs used in a mixture is less than 5 μM. In an aspect, the total concentration of all OFPs used in a mixture is less than 2.5 μM. In an aspect, the total concentration of all OFPs used in a mixture is less than 1 μM. In an aspect, the total concentration of all IFPs used in a mixture is less than 10 μM. In an aspect, the total concentration of all IFPs used in a mixture is less than 7.5 μM. In an aspect, the total concentration of all IFPs used in a mixture is less than 5 μM. In an aspect, the total concentration of all IFPs used in a mixture is less than 2.5 μM. In an aspect, the total concentration of all IFPs used in a mixture is less than 1 μM.

In an aspect, an IFP and an OFP do not form primer dimers.

In an aspect, a method comprises the introduction of at least one Universal Long Outer Forward Primer (ULOFP). In an aspect, the at least one ULOFP is added after the first polymerase extension step of a method. In an aspect, a method comprises the introduction of at least one Universal Long Reverse Primer (ULRP). In an aspect, the at least one ULRP is added after the first polymerase extension step of a method. In an aspect, a method comprises the introduction of at least one ULOFP and at least one ULRP. In an aspect, the at least one ULOFP and at least one ULRP are added after the first polymerase extension step of a method. In an aspect, a method comprises the introduction of a plurality of ULOFPs and a plurality of ULRPs. In an aspect, the plurality of ULOFPs and plurality of ULRPs are added after the first polymerase extension step of a method.

In an aspect, a ULOFP or a ULRP comprises a sequence that is at least 70% identical or complementary to the sequence of a universal adapter sequence. In an aspect, a ULOFP or a ULRP comprises a sequence that is at least 75% identical or complementary to the sequence of a universal adapter sequence. In an aspect, a ULOFP or a ULRP comprises a sequence that is at least 80% identical or complementary to the sequence of a universal adapter sequence. In an aspect, a ULOFP or a ULRP comprises a sequence that is at least 85% identical or complementary to the sequence of a universal adapter sequence. In an aspect, a ULOFP or a ULRP comprises a sequence that is at least 90% identical or complementary to the sequence of a universal adapter sequence. In an aspect, a ULOFP or a ULRP comprises a sequence that is at least 95% identical or complementary to the sequence of a universal adapter sequence. In an aspect, a ULOFP or a ULRP comprises a sequence that is at least 97.5% identical or complementary to the sequence of a universal adapter sequence. In an aspect, a ULOFP or a ULRP comprises a sequence that is at least 99% identical or complementary to the sequence of a universal adapter sequence. In an aspect, a ULOFP or a ULRP comprises a sequence that is 100% identical or complementary to the sequence of a universal adapter sequence.

In an aspect, a ULOFP comprises at least 25 nucleotides. In an aspect, a ULOFP comprises at least 30 nucleotides. In an aspect, a ULOFP comprises at least 35 nucleotides. In an aspect, a ULOFP comprises at least 40 nucleotides. In an aspect, a ULOFP comprises at least 50 nucleotides. In an aspect, a ULOFP comprises at least 60 nucleotides. In an aspect, a ULOFP comprises at least 70 nucleotides. In an aspect, a ULOFP comprises at least 80 nucleotides. In an aspect, a ULOFP comprises at least 90 nucleotides. In an aspect, a ULOFP comprises at least 100 nucleotides. In an aspect, a ULOFP comprises at least 110 nucleotides. In an aspect, a ULOFP comprises at least 125 nucleotides. In an aspect, a ULOFP comprises between 20 nucleotides and 125 nucleotides. In an aspect, a ULOFP comprises between 30 nucleotides and 100 nucleotides. In an aspect, a ULOFP comprises between 30 nucleotides and 75 nucleotides. In an aspect, a ULOFP comprises between 30 nucleotides and 50 nucleotides. In an aspect, a ULOFP comprises between 50 nucleotides and 100 nucleotides.

In an aspect, a ULRP comprises at least 25 nucleotides. In an aspect, a ULRP comprises at least 30 nucleotides. In an aspect, a ULRP comprises at least 35 nucleotides. In an aspect, a ULRP comprises at least 40 nucleotides. In an aspect, a ULRP comprises at least 50 nucleotides. In an aspect, a ULRP comprises at least 60 nucleotides. In an aspect, a ULRP comprises at least 70 nucleotides. In an aspect, a ULRP comprises at least 80 nucleotides. In an aspect, a ULRP comprises at least 90 nucleotides. In an aspect, a ULRP comprises at least 100 nucleotides. In an aspect, a ULRP comprises at least 110 nucleotides. In an aspect, a ULRP comprises at least 125 nucleotides. In an aspect, a ULRP comprises between 20 nucleotides and 125 nucleotides. In an aspect, a ULRP comprises between 30 nucleotides and 100 nucleotides. In an aspect, a ULRP comprises between 30 nucleotides and 75 nucleotides. In an aspect, a ULRP comprises between 30 nucleotides and 50 nucleotides. In an aspect, a ULRP comprises between 50 nucleotides and 100 nucleotides.

In an aspect, an IFP anneals to a template nucleic acid molecule at a temperature between 42° C., and 72° C. In an aspect, an IFP anneals to a template nucleic acid molecule at a temperature between 45° C., and 72° C. In an aspect, an IFP anneals to a template nucleic acid molecule at a temperature between 50° C., and 70° C. In an aspect, an IFP anneals to a template nucleic acid molecule at a temperature between 55° C., and 65° C.

In an aspect, an IFP comprises a single-stranded sequence at its 5′ end that does not bind (e.g., hybridize) to a template nucleic acid molecule. In an aspect, the single-stranded sequence comprises a sequencing adapter. In an aspect, the sequencing adapter is an adapter for adding an index adapter. In an aspect, a sequencing adapter is selected from the group consisting of an Illumina sequencing adapter, a Nanopore sequencing adapter, and an Ion Torrent sequencing adapter.

In an aspect, use of an OFP and an IFP provide a nested polymerase chain reaction (PCR) that further comprises a middle PCR to improve the specificity and on-target rate. In an aspect, a middle PCR comprises using an MFP that binds to an MFP binding site on at least one template nucleic acid molecule, wherein the MFP binding site partially overlaps with the OFP binding site, the IFP binding site, or both. In an aspect, a middle PCR comprises using an MFP that comprises a 5′ region starting from the second nucleotide of the OFP 5′ region to the second nucleotide of the OFP 3′ region, and the MFP comprises a 3′ region starting from the second nucleotide of the IFP 5′ region to the second nucleotide of the IFP 3′ region.

In an aspect, a method comprises a first set of primers comprising a first OFP and a first IFP and a second set of primers comprising a second OFP and a second IFP, and wherein between 0 nucleotides and 1000 nucleotides are positioned between the first set of primers and the second set of primers. In an aspect, a method comprises a first set of primers comprising a first OFP and a first IFP and a second set of primers comprising a second OFP and a second IFP, and wherein between 0 nucleotides and 750 nucleotides are positioned between the first set of primers and the second set of primers. In an aspect, a method comprises a first set of primers comprising a first OFP and a first IFP and a second set of primers comprising a second OFP and a second IFP, and wherein between 0 nucleotides and 500 nucleotides are positioned between the first set of primers and the second set of primers. In an aspect, a method comprises a first set of primers comprising a first OFP and a first IFP and a second set of primers comprising a second OFP and a second IFP, and wherein between 0 nucleotides and 250 nucleotides are positioned between the first set of primers and the second set of primers. In an aspect, a method comprises a first set of primers comprising a first OFP and a first IFP and a second set of primers comprising a second OFP and a second IFP, and wherein between 0 nucleotides and 100 nucleotides are positioned between the first set of primers and the second set of primers. In an aspect, a method comprises a first set of primers comprising a first OFP and a first IFP and a second set of primers comprising a second OFP and a second IFP, and wherein between 0 nucleotides and 50 nucleotides are positioned between the first set of primers and the second set of primers. In an aspect, a method comprises a first set of primers comprising a first OFP and a first IFP and a second set of primers comprising a second OFP and a second IFP, and wherein between 0 nucleotides and 25 nucleotides are positioned between the first set of primers and the second set of primers.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

The terms “percent complementarity” or “percent complementary” as used herein in reference to two nucleotide sequences refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of complementary nucleotide bases, such as guanine (G)-cytosine (C), adenine (A)-thymine (T), and A-uracil (U), through hydrogen binding. If the “percent complementarity.” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

As used herein, a “portion” of a nucleic acid molecule refers to contiguous set of nucleotides comprised by that molecule. A portion can comprise all or only a subset of the nucleotides comprised by the molecule. A portion can be double-stranded or single-stranded.

As used herein, a “mismatch” refers to an alignment of two sequences that pairs two uncomplimentary nucleotides. Non-limiting examples of mismatches include G-A, G-T, G-U, G-G, C-A, C-T, C-U, C-C, A-A, T-T, and T-U. Conversely, “matched” alignments of nucleotides refer to complimentary pairs such as G-C, A-T, and A-U.

As a non-limiting example, the complement of the sequence 5′-ATGC-3′ is 3′-TACG-5′, and the reverse complement of 5′-ATGC-3′ is 5′-GCAT-3′. Notably, the complement and reverse complement sequences are identical to each other when viewed in the 5′ to 3′ direction.

For optimal alignment of sequences to calculate their percent complementarity, various pair-wise or multiple sequence alignment algorithms and programs are known in the art, such as ClustalW or Basic Local Alignment Search Tool® (BLAST™), etc., that can be used to compare the sequence complementarity or identity between two or more nucleotide sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31:3497-3500 (2003): Thompson et al., “Clustal W: Improving the sensitivity of progressive multiple sequence alignment through sequence weighting, position-specific gap penalties and weight matrix choice,” Nucleic Acids Research 22:4673-4680 (1994): Larkin M A et al., “Clustal W and Clustal X version 2.0,” Bioinformatics 23:2947-48 (2007); and Altschul et al. “Basic local alignment search tool.” J. Mol. Biol. 215:403-410 (1990), the entire contents and disclosures of which are incorporated herein by reference. For alignment between fusion sequences, alternative programs such as STAR or STAR-Fusion can be used. See Jia et al., Genome Biol. 14: R12 (2013): Haas et al. Genome Biol. 20:213 (2019).

In one aspect, this disclosure provides a method for detecting at least one gene fusion in a test sample, the method comprising: (a) obtaining DNA extracted from a cell line or a clinical patient sample, or obtaining cDNA generated from RNA extracted from a cell line or a clinical patent sample to generate the test sample: (b) introducing to the test sample: (i) at least one Randomer Reverse Primer (RRP), where the at least one RRP comprises a Randomer Sequence (RS) positioned on the 3′ end of the at least one RRP and a first universal adapter sequence positioned 5′ to the RS: (ii) at least one Outer Forward Primer (OFP) that binds to an OFP binding site: (iii) a first polymerase; and (iv) a reaction buffer to create a first mixture: (c) subjecting the first mixture to a first polymerase extension step to generate at least one extended product: (d) diluting or purifying the at least one extended product to generate a second mixture comprising the at least one extended product: (e) introducing to the second mixture (i) at least one Inner Forward Primer (IFP) that binds to an IFP binding site: (ii) a universal reverse primer: (iii) a thermostable polymerase; and (iv) reagents for thermostable polymerase activity to generate a third mixture: (f) subjecting the third mixture to a second polymerase extension step to generate at least one amplicon from the at least one extended product; and (g) analyzing the at least one amplicon to identify an amplicon comprising the at least one gene fusion. In an aspect, step (g) further comprises generating sequencing reads of the at least one amplicon using next generation sequencing. In an aspect, the method further comprises repeating steps (a) to (g) after adjusting the concentration of the at least one IFP to IFP_{new}, wherein IFP_{new}=IFP_{old}*(Reads_median/Reads_amplicon)^{X}, wherein IFP_{old} is the concentration of the at least one IFP in the first iteration of step (e): Reads_median is the median reads mapped to each amplicon: Reads_amplicon is the reads mapped to the amplicon corresponding to said forward primer; and X is an adjustment factor between 0.25 and 1.

As used herein, a “gene fusion” refers to a hybrid gene that is formed from two previously independent genes. Without being limiting, gene fusions can result from a translocation, an interstitial deletion, or a chromosomal inversion. When two genes fuse, the point of fusion is called a “breakpoint.” In an aspect, a breakpoint is positioned between an exon from a first gene and an exon from a second gene. In an aspect, a breakpoint is positioned between an exon from a first gene and an intron from a second gene. In an aspect, a breakpoint is positioned between an intron from a first gene and an intron from a second gene. In an aspect, a breakpoint is positioned between a 5′ untranslated region (UTR), a 3′ UTR, or a promoter of a first gene and a 5′ UTR, a 3′ UTR, a promoter, an exon, or an intron of a second gene. In an aspect, a gene fusion is a DNA fusion. In an aspect, a gene fusion is an RNA fusion.

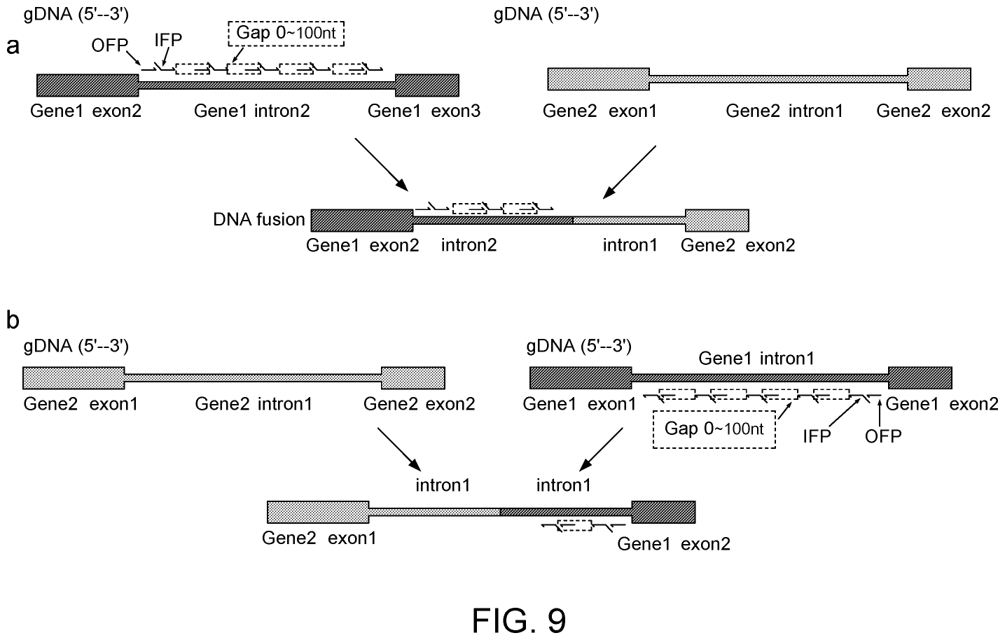

Methods provided herein that use RRPs can be used to detect DNA fusion. A DNA fusion typically, but not always, has a breakpoint in an intron. Therefore, designing primers that cover entire intron regions are necessary to detect unknown DNA fusions. See, for example, FIG. 9.

In an aspect, a test sample comprises at least one gene fusion. In an aspect, a test sample comprises a plurality of gene fusions. In an aspect, a test sample does not comprise a gene fusion.

In an aspect, an IFP binding site is positioned between 0 nucleotides and 250 nucleotides from a breakpoint of a gene fusion. In an aspect, an IFP binding site is positioned between 0 nucleotides and 100 nucleotides from a breakpoint of a gene fusion. In an aspect, an IFP binding site is positioned between 0 nucleotides and 75 nucleotides from a breakpoint of a gene fusion. In an aspect, an IFP binding site is positioned between 0 nucleotides and 50 nucleotides from a breakpoint of a gene fusion. In an aspect, an IFP binding site is positioned between 0 nucleotides and 40 nucleotides from a breakpoint of a gene fusion. In an aspect, an IFP binding site is positioned between 0 nucleotides and 30 nucleotides from a breakpoint of a gene fusion. In an aspect, an IFP binding site is positioned between 0 nucleotides and 20 nucleotides from a breakpoint of a gene fusion. In an aspect, an IFP binding site is positioned between 0 nucleotides and 10 nucleotides from a breakpoint of a gene fusion.

In an aspect, an amplicon comprises a target exon sequence. As used herein, a “target exon sequence” refers to an exon sequence that is desired to be amplified. In an aspect, a target exon sequence comprises a breakpoint of a gene fusion.

In an aspect, the 3′ end of at least one IFP binds to an IFP binding site which is between 0 nucleotides and 10 nucleotides from the 3′ end of a target exon sequence for detecting a gene fusion. In an aspect, the 3′ end of at least one IFP binds to an IFP binding site which is between 0 nucleotides and 20 nucleotides from the 3′ end of a target exon sequence for detecting a gene fusion. In an aspect, the 3′ end of at least one IFP binds to an IFP binding site which is between 0 nucleotides and 30 nucleotides from the 3′ end of a target exon sequence for detecting a gene fusion. In an aspect, the 3′ end of at least one IFP binds to an IFP binding site which is between 0 nucleotides and 40 nucleotides from the 3′ end of a target exon sequence for detecting a gene fusion. In an aspect, the 3′ end of at least one IFP binds to an IFP binding site which is between 0 nucleotides and 50 nucleotides from the 3′ end of a target exon sequence for detecting a gene fusion.

In an aspect, a method comprises the use of a first IFP and a second IFP, and wherein between 0 nucleotides and 25 nucleotides are positioned between the 3′ end of the first IFP and the 5′ end of the second IFP. In an aspect, a method comprises the use of a first IFP and a second IFP, and wherein between 0 nucleotides and 50 nucleotides are positioned between the 3′ end of the first IFP and the 5′ end of the second IFP. In an aspect, a method comprises the use of a first IFP and a second IFP, and wherein between 0 nucleotides and 100 nucleotides are positioned between the 3′ end of the first IFP and the 5′ end of the second IFP. In an aspect, a method comprises the use of a first IFP and a second IFP, and wherein between 0 nucleotides and 200 nucleotides are positioned between the 3′ end of the first IFP and the 5′ end of the second IFP. In an aspect, a method comprises the use of a first IFP and a second IFP, and wherein between 0 nucleotides and 300 nucleotides are positioned between the 3′ end of the first IFP and the 5′ end of the second IFP. In an aspect, a method comprises the use of a first IFP and a second IFP, and wherein between 0 nucleotides and 500 nucleotides are positioned between the 3′ end of the first IFP and the 5′ end of the second IFP.

In an aspect, an IFP enables the identification of all exons involved in a gene fusion. In an aspect, an IFP enables the identification of all introns involved in a gene fusion.

In an aspect, this disclosure provides a method of designing a plurality of Inner Forward Primers (IFPs) and a plurality of Outer Forward Primers (OFPs) to identify a gene fusion, within the gene of interest, where an adjacent IFP and OFP designed to amplify the same target region of the gene of interest form a primer set, and where: (a) the 3′ end of an OFP binding site and the 5′ end of an IFP binding site overlap for a primer set: (b) at least one nucleotide is positioned between the 3′ end of an OFP binding site and the 5′ end of an IFP binding site for a primer set: or (c) zero nucleotides are positioned between the 3′ end of an OFP binding site and the 5′ end of the IFP binding site, and the OFP binding site and an IFP binding site do not overlap for a primer set: where the IFPs and OFPs are used to identify a gene fusion in the nucleic acid molecule. In an aspect, the method further comprises generating at least one amplicon of the nucleic acid molecule using the OFPs and IFPs via isothermal extension, PCR, or both.

In an aspect, an IFP is designed to hybridize to the positive strand of a reference sequence of a nucleic acid molecule. In an aspect, an OFP is designed to hybridize to the positive strand of a reference sequence of a nucleic acid molecule. In an aspect, an IFP is designed to hybridize to the negative strand of a reference sequence of a nucleic acid molecule. In an aspect, an OFP is designed to hybridize to the negative strand of a reference sequence of a nucleic acid molecule.

In an aspect, a method comprises designing a plurality of IFPs for the forward strand of a nucleic acid molecule. In an aspect, a method comprises designing a plurality of OFPs for the forward strand of a nucleic acid molecule. In an aspect, a method comprises designing a plurality of IFPs for the reverse strand of a nucleic acid molecule. In an aspect, a method comprises designing a plurality of OFPs for the reverse strand of a nucleic acid molecule.

In an aspect, a plurality of IFPs are designed to enable the identification of all exons involved in a gene fusion. In an aspect, a plurality of IFPs are designed to enable the identification of all introns involved in a gene fusion.

In an aspect, a plurality of IPFs and a plurality of OFPs are designed to tile an entire exon region of a gene fusion. In an aspect, a plurality of IPFs and a plurality of OFPs are designed to tile an entire intron region of a gene fusion.

In an aspect, this disclosure provides a method for detecting alternative RNA splicing, the method comprising: (a) introducing to a sample comprising cDNA generated from an RNA sample comprising at least one RNA splicing variant: (i) at least one Randomer Reverse Primer (RRP), where the at least one RRP comprises a Randomer Sequence (RS) positioned on the 3′ end of the at least one RRP and a first universal adapter sequence positioned 5′ to the RS: (ii) a first polymerase; and (iii) a reaction buffer to create a first mixture: (b) subjecting the first mixture to a first polymerase extension step to generate a second mixture comprising at least one extended product: (c) purifying the at least one extended product from step (b) to generate a second mixture comprising the at least one extended product: (d) introducing to the second mixture: (i) at least one Inner Forward Primer (IFP) that binds to an IFP binding site on the at least one nucleic acid template molecule: (ii) a second adapter sequence: (iii) a thermostable polymerase; and (iv) reagents for thermostable polymerase activity to generate a third mixture: (e) subjecting the third mixture to thermal cycling to generate at least one amplicon of the at least one extended product; and (f) analyzing the at least one amplicon to identify the at least one RNA splicing variant. In an aspect, the at least one amplicon is purified following step (f).

In an aspect, an OFP binds to at least one exon of a gene comprising at least one RNA splicing variant. In an aspect, an OFP, or a plurality of OFPs, binds to every exon of a gene comprising at least one RNA splicing variant.

In an aspect, an index primer for sequencing is added to any amplicon provided herein in.

In an aspect, sequencing an amplicon, or a plurality of amplicons, is performed using next-generation sequencing technologies. In an aspect, sequencing an amplicon, or a plurality of amplicons, is performed using a sequencing instrument selected from the group consisting of an Oxford Nanopore sequencer, a PacBio sequencer, an Illumina Miseq sequencer, an Illumina MiniSeq sequencer, an Illumina NextSeq sequencer, an Ion Torrent sequencer, and an Illumina Hiseq sequencer to generate at least one sequencing read. In an aspect, a sequencing read is aligned to a reference sequence to identify an RNA splicing variant.

The following exemplary, non-limiting, embodiments are envisioned:

1. A method of preparing a sequencing library, the method comprising:
(a) introducing to a sample comprising at least one nucleic acid template molecule:
   (i) at least one Randomer Reverse Primer (RRP), wherein the at least one RRP comprises a Randomer Sequence (RS) positioned within the 3′ region of the at least one RRP and a first universal adapter sequence positioned 5′ to the RS;
   (ii) a first polymerase; and
   (iii) a reaction buffer
to create a first mixture;
(b) subjecting the first mixture to a first polymerase extension step to generate a second mixture comprising at least one extended product;
(c) introducing to the second mixture:
   (i) at least one Inner Forward Primer (IFP) that binds to an IFP binding site on the at least one nucleic acid template molecule;
   (ii) a second adapter sequence;
   (iii) a thermostable polymerase; and
   (iv) one or more reagents for thermostable polymerase activity to generate a third mixture; and
(d) subjecting the third mixture to a second polymerase extension step to generate at least one amplicon of the at least one nucleic acid template molecule.

2. A method of preparing a sequencing library, the method comprising:
(a) introducing to a sample comprising at least one nucleic acid template molecule:
   (i) at least one Randomer Reverse Primer (RRP), wherein the at least one RRP comprises a Randomer Sequence (RS) positioned on the 3′ region of the at least one RRP and a first universal adapter sequence positioned 5′ to the RS;
   (ii) at least one Outer Forward Primer (OFP);
   (iii) a first polymerase; and
   (iv) a reaction buffer
to create a first mixture;
(b) subjecting the first mixture to a first polymerase extension step to generate a second mixture comprising at least one extended product;
(c) introducing to the second mixture:
   (i) at least one Inner Forward Primer (IFP) that binds to an IFP binding site on the at least one nucleic acid template molecule;
   (ii) a second adapter sequence;
   (iii) a thermostable polymerase, and
   (iv) one or more reagents for thermostable polymerase activity to generate a third mixture; and
(d) subjecting the third mixture to a second polymerase extension step to generate at least one amplicon of the at least one nucleic acid template molecule.

3. The method of embodiment 1 or 2, wherein the first polymerase extension step comprises isothermal extension.

4. The method of embodiment 1, wherein the method further comprises introducing to the sample at least one Outer Forward Primer (OFP) in step (a).

5. The method of embodiment 2 or 4, wherein the first polymerase extension step comprises thermal cycling.

6. The method of embodiment 1 or 2, wherein the second mixture is diluted between step (b) and step (c).

7. The method of embodiment 6, wherein the second mixture is diluted at a ratio between 1:10 and 1:10,000.

8. The method of embodiment 1 or 2, wherein the at least one extended product is purified from the second mixture between step (b) and step (c).

9. The method of embodiment 8, wherein the at least one extended product is purified using a technique selected from the group consisting of column purification and beads purification.

10. The method of embodiment 1 or 2, wherein the first universal adapter sequence is positioned at the 5′ end of the RRP.

11. The method of any one of embodiments 1-10, wherein the RS comprises at least two degenerate nucleotides selected from the group consisting of R, Y, S, W, K, M, B, D, H, V, and N.

12. The method of any one of embodiments 1-11, wherein the first universal adapter sequence is an Adapter_2 sequence.

13. The method of any one of embodiments 1-12, wherein the second adapter sequence is an Adapter_3 sequence.

14. The method of embodiments 1-13, wherein the at least one RRP further comprises a Non-Relevant Sequence (NRS).

15. The method of embodiment 14, wherein the NRS comprises less than 50% complementarity with the at least one nucleic acid template molecule.

16. The method of embodiment 14, wherein the NRS comprises less than 50% complementarity to the first universal adapter sequence, the second adapter sequence, or both.

17. The method of any one of embodiments 14-16, wherein the NRS comprises between 5 nucleotides and 20 nucleotides.

18. The method of any one of embodiments 14-17, wherein the NRS is positioned 5′ to the RS and 3′ to the first universal adapter sequence.

19. The method of any one of embodiments 1-18, wherein the RS comprises between 3 nucleotides and 30 nucleotides.

20. The method of any one of embodiments 1-19, wherein the at least one RRP further comprises a Mixture of Specific Sequences (MSS) at the 3′ end of the at least one RRP.

21. The method of embodiment 20, wherein the MSS comprises between 2 nucleotides and 5 nucleotides.

22. The method of embodiment 20 or 21, wherein the MSS is comprised of A, T, G, and/or C nucleotides.

23. The method of any one of embodiments 1-22, wherein the at least one RRP further comprises a Unique Molecular Identifier (UMI) sequence.

24. The method of embodiment 23, wherein the UMI sequence is positioned 5′ to the RS sequence and 3′ to the first universal adapter sequence.

25. The method of embodiment 23 or 24, wherein the UMI sequence comprises a mixture of between 10 and 100 defined DNA sequences with a minimum pairwise Hamming distance of between 2 and 5.

26. The method of embodiment 23 or 24, wherein the UMI sequence comprises a mixture of between 10 and 1000 defined DNA sequences with a minimum pairwise Levenschtein distance of between 2 and 5.

27. The method of any one of embodiments 23-26, wherein the UMI sequence comprises at least one degenerate nucleotide selected from the group consisting of R, Y, S, W, K, M, B, D, H, V, and N.

28. The method of any one of embodiments 1-27, wherein the at least one RRP further comprises a Sample Barcode (SB) sequence.

29. The method of embodiment 28, wherein the SB sequence is positioned 5′ to the RS sequence and 3′ to the first universal adapter sequence.

30. The method of any one of embodiments 1-29, wherein the at least one nucleic acid template molecule is a DNA template molecule.

31. The method of any one of embodiments 1-29, wherein the at least one nucleic acid template molecule is an RNA template molecule.

32. The method of embodiment 1 or 2, wherein the first polymerase, the thermostable polymerase, or both, is an RNA polymerase.

33. The method of embodiment 1 or 2, wherein the first polymerase, the thermostable polymerase, or both, is a DNA polymerase.

34. The method of embodiment 1 or 2, wherein the first polymerase is selected from the group consisting of phi29 DNA polymerase, DNA polymerase 1, large (Klenow) fragment, Klenow fragment, Bst DNA polymerase, T4 DNA polymerase, T7 DNA polymerase, Taq polymerase, Phusion® polymerase, Q5® polymerase, KAPA HiFi polymerase, Vent® DNA polymerase, LongAmp® Taq DNA polymerase, and OneTaq® DNA polymerase.

35. The method of embodiment 1 or 2, wherein the first polymerase, the thermostable polymerase, or both, is selected from the group consisting of Taq polymerase, Phusion® polymerase, Q5® polymerase, KAPA HiFi polymerase, Vent® DNA polymerase, LongAmp® Taq DNA polymerase, and OneTaq® DNA polymerase.

36. The method of embodiment 1 or 2, wherein the first polymerase is selected from the group consisting of a phi29 DNA polymerase, a Bst DNA polymerase, a Bst 2.0 DNA polymerase, a Bst 3.0 DNA polymerase, a T7 DNA polymerase, a T4 DNA polymerase, and a DNA polymerase 1 large (Klenow) fragment.

37. The method of embodiment 1 or 2, wherein the thermostable polymerase is selected from the group consisting of Taq polymerase, Phusion® polymerase, Q5R polymerase, KAPA HiFi polymerase, Vent® DNA polymerase, LongAmp® Taq DNA polymerase, and OneTaq® DNA polymerase.

38. The method of embodiment 3, wherein the isothermal extension occurs at a temperature between 20° C., and 65° C.

39. The method of embodiment 1 or 2, wherein the first polymerase extension step comprises a duration of between 10 seconds and 6 hours.

40. The method of embodiment 2, wherein the first polymerase extension step comprises thermal cycling.

41. The method of embodiment 40, wherein the thermal cycling comprises an annealing temperature of between 45° C., and 72° C.

42. The method of embodiment 1 or 2, wherein the second polymerase extension step comprises thermal cycling, and wherein the thermal cycling comprises an annealing temperature of between 45° C., and 72° C.

43. The method of any one of embodiments 40-42, wherein the thermal cycling comprises between 1 and 50 thermal cycles.

44. The method of embodiment 2 or 4, wherein the method further comprises the introduction of at least one Universal Long Outer Forward Primer (ULOFP), at least one Universal Long Reverse Primer (ULRP), or both.

45. The method of embodiment 2 or 4, wherein the at least one OFP comprises a Gene Specific Outer Forward Primer (GSOFP) that binds to a GSOFP binding site on the at least one template nucleic acid molecule, and a second universal adapter sequence.

46. The method of embodiment 45, wherein the at least one ULOFP comprises at least 70% homology to the second universal adapter sequence.

47. The method of embodiment 44, wherein the at least one ULRP comprises at least 70% homology to the first universal adapter sequence.

48. The method of embodiment 44, wherein the at least one ULOFP comprises between 30 nucleotides and 100 nucleotides.

49. The method of embodiment 44, wherein the at least one ULRP comprises between 30 nucleotides and 100 nucleotides.

50. The method of embodiment 45, wherein the GSOFP comprises between 10 nucleotides and 70 nucleotides.

51. The method of any one of embodiments 1-50, wherein the at least one amplicon is purified from the third mixture.

52. The method of embodiment 51, wherein the at least one amplicon is purified from the third mixture using column purification or beads purification.

53. The method of any one of embodiments 1-50, wherein the at least one amplicon is diluted at a ratio of between 1:10 and 1:10,000.

54. The method of any one of embodiments 1-53, wherein the at least one IFP comprises between 10 nucleotides and 70 nucleotides.

55. The method of any one of embodiments 1-54, wherein the at least one IFP anneals to the at least one template nucleic acid molecule at a temperature between 45° C., and 72° C.

56. The method of any one of embodiments 1-55, wherein the at least one IFP comprises a single-stranded sequence at its 5′ end that does not bind to the at least one template nucleic acid molecule.

57. The method of embodiment 56, wherein the single-stranded sequence comprises a sequencing adapter.

58. The method of embodiment 57, wherein the sequencing adapter is an adapter for adding an index adapter.

59. The method of embodiment 57, wherein the sequencing adapter is selected from the group consisting of an Illumina sequencing adapter, a Nanopore sequencing adapter, and an Ion Torrent sequencing adapter.

60. The method of embodiment 45, wherein the IFP binding site is positioned 5′ on the at least one nucleic acid template molecule as compared to the GSOFP binding site.

61. The method of any one of embodiments 1-60, wherein the at least one IFP comprises at least one Gene Specific Inner Forward Primer (GSIFP) sequence, wherein the GSIFP sequence binds to the at least one template nucleic acid molecule at a GSIFP binding site.

62. The method of embodiment 45, wherein the IFP binding site and the GSOFP binding site overlap on the 5′ end of the IFP binding site and the 3′ end of the GSOFP binding site.

63. The method of embodiment 62, wherein the overlap comprises between 1 nucleotide and 40 nucleotides.

64. The method of embodiment 45, wherein between 1 nucleotide and 50 nucleotides are positioned between the 3′ end of the GSOFP binding site and the 5′ end of the IFP binding site.

65. The method of embodiment 64, wherein the IFP binding site is a GSIFP binding site.

66. The method of embodiment 45, wherein the IFP binding site and the GSOFP binding site are adjacent.

67. The method of embodiment 66, wherein the IFP binding site is a GSIFP binding site.

68. The method of any one of embodiments 1-59, wherein the at least one OFP and at least one IFP provide a nested PCR that further comprises a middle PCR to improve the specificity and on-target rate.

69. The method of embodiment 68, wherein the middle PCR comprises using a Middle Forward Primer (MFP) that binds to an MFP binding site on the at least one template nucleic acid molecule, and wherein the MFP binding site partially overlaps with the OFP binding site, the IFP binding site, or both.

70. The method of embodiment 68, wherein the middle PCR comprises using a Middle Forward Primer (MFP) that comprises a 5′ region starting from the second nucleotide of the OFP 5′ region to the second nucleotide of the OFP 3′ region, and the MFP comprises a 3′ region starting from the second nucleotide of the IFP 5′ region to the second nucleotide of the IFP 3′ region.

71. The method of embodiment 69 or 70, wherein the MFP comprises between 10 nucleotides and 70 nucleotides.

72. The method of any one of embodiments 1-71, wherein at least one template molecule comprises at least part of one exon, at least part of one intron, or both.

73. The method of any one of embodiments 1-71, wherein the at least one template molecule comprises at least one long region of interest having a length of at least 50 nucleotides.

74. The method of any one of embodiments 1-73, wherein the method comprises the use of a plurality of IFPs and a plurality of OFPs.

75. The method of embodiment 73, wherein a plurality of IFPs are used to tile the long region of interest.

76. The method of embodiment 75, wherein the tile is conducted in two orientations, one of which is based on the positive strand of the template nucleic acid molecule, and the other of which is based on the negative strand of the template nucleic acid molecule.

77. The method of any one of embodiments 1-73, wherein the IFP binding site is positioned between 0 nucleotides and 20 nucleotides from a breakpoint of a gene fusion.

78. The method of embodiment 74, wherein the method comprises a first set of primers comprising a first OFP and a first IFP and a second set of primers comprising a second OFP and a second IFP, and wherein between 0 nucleotides and 100 nucleotides are positioned between the first set of primers and the second set of primers.

79. The method of any one of embodiments 1-78, wherein the thermal cycling further comprises the use of at least one wildtype-specific blocker.

80. The method of embodiment 79, wherein the at least one wildtype-specific blocker corresponds to a wildtype sequence that overlaps with the at least one IFP by between 2 nucleotides and 30 nucleotides.

81. The method of embodiment 80, wherein:
(a) the overlap comprises a standard free energy of binding between −2 kcal/mol and −4 kcal/mol;
(b) sequence of the at least one IFP that does not overlap with the at least one wildtype-specific blocker comprises a standard free energy between −5 kcal/mol and −9 kcal/mol; and
(c) sequence of the at least one wildtype-specific blocker that does not overlap with the at least one IFP comprises a standard free energy between −7 kcal/mol and −12 kcal/mol.

82. The method of any one of embodiments 79-81, wherein the at least one wildtype-specific blocker comprises a terminator to prevent 3′ to 5′ DNA polymerase exonuclease activity.

83. The method of embodiment 82, wherein the terminator is selected from the group consisting of a C3 spacer and DXXDM.

84. The method of any one of embodiments 1-83, wherein the at least one or at least two IFPs are present at a concentration of between 1 nM and 1000 nM.

85. The method of any one of embodiments 1-83, wherein the at least one or at least two OFPs are present at a concentration of between 1 nM and 1000 nM.

86. A method for detecting at least one gene fusion in a test sample, the method comprising:
(a) obtaining DNA extracted from a cell line or a clinical patient sample, or obtaining cDNA generated from RNA extracted from a cell line or a clinical patent sample to generate the test sample;
(b) introducing to the test sample:
   (i) at least one Randomer Reverse Primer (RRP), wherein the at least one RRP comprises a Randomer Sequence (RS) positioned on the 3′ end of the at least one RRP and a first universal adapter sequence positioned 5′ to the RS;
   (ii) at least one Outer Forward Primer (OFP) that binds to an OFP binding site;
   (iii) a first polymerase; and
   (iv) a reaction buffer
to create a first mixture;
(c) subjecting the first mixture to a first polymerase extension step to generate at least one extended product;
(d) diluting or purifying the at least one extended product to generate a second mixture comprising the at least one extended product;
(e) introducing to the second mixture
   (i) at least one Inner Forward Primer (IFP) that binds to an IFP binding site;
   (ii) a universal reverse primer;
   (iii) a thermostable polymerase; and
   (iv) one or more reagents for thermostable polymerase activity to generate a third mixture;
(f) subjecting the third mixture to a second polymerase extension step to generate at least one amplicon from the at least one extended product; and
(g) analyzing the at least one amplicon to identify an amplicon comprising the at least one gene fusion.

87. The method of embodiment 86, wherein the test sample comprises at least one gene fusion.

88. The method of embodiment 86, wherein the test sample does not comprise a gene fusion.

89. The method of embodiment 86, wherein the IFP binding site, the OFP binding site, or both, is on the positive strand of the DNA or cDNA.

90. The method of embodiment 86, wherein the IFP binding site, the OFP binding site, or both, is on the negative strand of the DNA or cDNA.

91. The method of embodiment 86, wherein the at least one amplicon comprises a target exon sequence.

92. The method of embodiment 91, wherein the 3′ end of the at least one IFP binds to an IFP binding site which is between 0 nucleotides and 20 nucleotides from the 3′ end of a target exon sequence for detecting a gene fusion.

93. The method of embodiment 86, wherein the method comprises the use of a first IFP and a second IFP, and wherein between 0 nucleotides and 100 nucleotides are positioned between the 3′ end of the first IFP and the 5′ end of the second IFP.

94. The method of embodiment 86, wherein the at least one IFP, the at least one OFP, or both, comprises a length of between 10 nucleotides and 100 nucleotides.

95. The method of any one of embodiments 86-94, wherein the at least one IFP, the at least one OFP, or both, comprises a standard free energy between −11.5 kcal/mol and −12.5 kcal/mol in a standard PCR buffer.

96. The method of any one of embodiments 86-95, wherein the at least one IFP, the at least one OFP, or both, does not form primer dimers.

97. The method of any one of embodiments 86-96, wherein the concentration of the at least one IFP, at least one OFP, or both, is between 0.1 nM and 1000 nM.

98. The method of any one of embodiments 86-97, wherein the total concentration of all OFPs and IFPs is less than 10 μM.

99. The method of embodiment 86, wherein step (g) comprises generating sequencing reads of the at least one amplicon using next generation sequencing.

100. The method of embodiment 99, wherein the method further comprises repeating steps (a) to (g) after adjusting the concentration of the at least one IFP to IFP_{new}, wherein IFP_{new}=IFP_{old}*(Reads_median/Reads_amplicon)^{X}, wherein IFP_{old} is the concentration of the at least one IFP in the first iteration of step (e); Reads_median is the median reads mapped to each amplicon: Reads_amplicon is the reads mapped to the amplicon corresponding to said forward primer; and X is an adjustment factor between 0.25 and 1.

101. A method of designing a plurality of Inner Forward Primers (IFPs) and a plurality of Outer Forward Primers (OFPs) to identify a gene fusion, within the gene of interest, wherein an adjacent IFP and OFP designed to amplify the same target region of the gene of interest form a primer set, and wherein:
(a) the 3′ end of an OFP binding site and the 5′ end of an IFP binding site overlap for a primer set;
(b) at least one nucleotide is positioned between the 3′ end of an OFP binding site and the 5′ end of an IFP binding site for a primer set: or
(c) zero nucleotides are positioned between the 3′ end of an OFP binding site and the 5′ end of the IFP binding site, and the OFP binding site and an IFP binding site do not overlap for a primer set;
wherein the IFPs and OFPs are used to identify a gene fusion in the nucleic acid molecule.

102. The method of embodiment 101, wherein the IFPs, the OFPs, or both, are designed to hybridize to the positive strand of a reference sequence of the nucleic acid molecule.

103. The method of embodiment 101, wherein the IFPs, the OFPs, or both, are designed to hybridize to the negative strand of a reference sequence of the nucleic acid molecule.

104. The method of embodiment 101 or 102, wherein the IFPs, the OFPs, or both, comprise a length of between 10 nucleotides and 100 nucleotides.

105. The method of any one of embodiments 101-104, wherein the IFPs, the OFPs, or both, comprise a standard free energy between −11.5 kcal/mol and −12.5 kcal/mol in a standard PCR buffer.

106. The method of any one of embodiments 101-105, wherein the IFPs, the OFPs, or both, do not form primer dimers.

107. The method of embodiment 101, wherein the method comprises designing IFPs, OFPs, or both, for the forward strand and the reverse strand of the nucleic acid molecule.

108. The method of any one of embodiments 101-107, wherein the IFPs enable 108, identification of all exons involved in the gene fusion.

109. The method of any one of embodiments 101-108, wherein the IFPs enable identification of all introns involved in the gene fusion.

110. The method of any one of embodiments 101-109, wherein the nucleic acid molecule is a DNA molecule.

111. The method of any one of embodiments 101-108, wherein the nucleic acid molecule is an RNA molecule.

112. The method of any one of embodiments 101-111, wherein the IFPs and OFPs tile an entire intron region of the gene fusion.

113. The method of any one of embodiments 101-112, wherein the IFPs and OFPs tile an entire exon region of the gene fusion.

114. The method of any one of embodiments 101-113, wherein the method further comprises generating at least one amplicon of the nucleic acid molecule using the OFPs and IFPs via isothermal extension, PCR, or both.

115. A method for detecting alternative RNA splicing, the method comprising:
(a) introducing to a sample comprising cDNA generated from an RNA sample comprising at least one RNA splicing variant:
   (i) at least one Randomer Reverse Primer (RRP), wherein the at least one RRP comprises a Randomer Sequence (RS) positioned on the 3′ end of the at least one RRP and a first universal adapter sequence positioned 5′ to the RS;
   (ii) a first polymerase; and
   (iii) a reaction buffer
to create a first mixture;
(b) subjecting the first mixture to a first polymerase extension step to generate a second mixture comprising at least one extended product;
(c) purifying the at least one extended product from step (b) to generate a second mixture comprising the at least one extended product;
(d) introducing to the second mixture:
   (i) at least one Inner Forward Primer (IFP) that binds to an IFP binding site on the at least one nucleic acid template molecule;
   (ii) a second adapter sequence;
   (iii) a thermostable polymerase; and
   (iv) one or more reagents for thermostable polymerase activity to generate a third mixture:
(e) subjecting the third mixture to thermal cycling to generate at least one amplicon of the at least one extended product; and
(f) analyzing the at least one amplicon to identify the at least one RNA splicing variant.

116. The method of embodiment 115, wherein the at least one OFP targets at least one exon of a gene comprising at least one RNA splicing variant.

117. The method of embodiment 115 or 116, wherein the at least one OFP targets every exon of a gene comprising the at least one RNA splicing variant.

118. The method of any one of embodiments 115-117, wherein the at least one amplicon is purified following step (f).

119. The method of embodiment 118, wherein an index primer for sequencing is added to the at least one amplicon.

The method of embodiment 119, wherein the method further comprises 120, sequencing the at least one amplicon using a sequencing instrument selected from the group consisting of an Oxford Nanopore sequencer, a PacBio sequencer, an Illumina Miseq sequencer, an Illumina MiniSeq sequencer, an Illumina NextSeq sequencer, an Ion Torrent sequencer, and an Illumina Hiseq sequencer to generate at least one sequencing read.

121. The method of embodiment 120, wherein the at least one sequencing read is aligned to a reference sequence to identify the at least one RNA splicing variant.

Having now generally described the disclosure, the same will be more readily understood through reference to the following examples that are provided by way of illustration, and are not intended to be limiting of the present disclosure, unless specified.

### Example 1. Design of Multiplex OFPs and IFPs

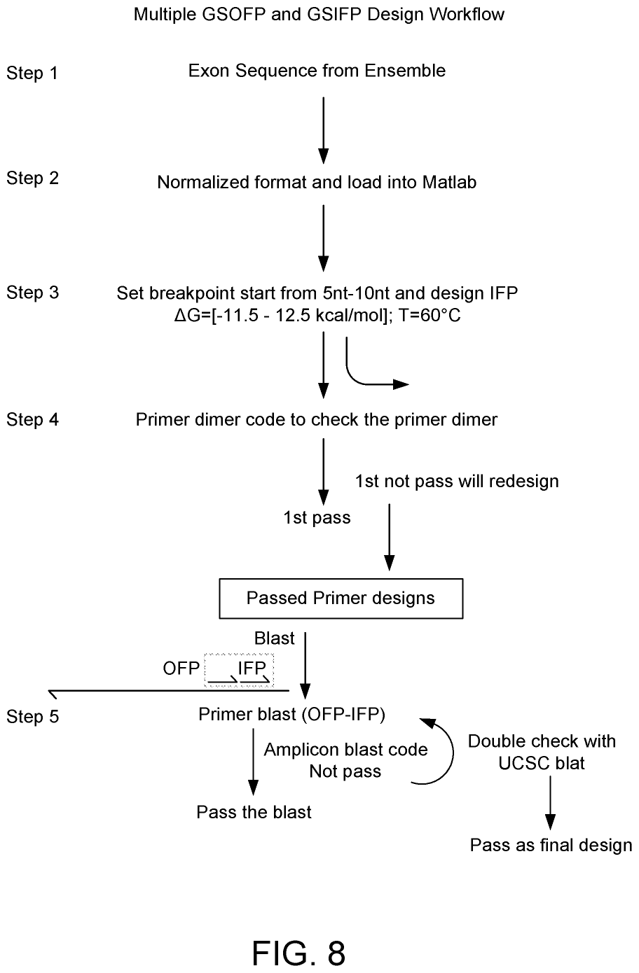

Without being limiting, FIG. 8 provides a schematic showing rules for designing multiplex GSOFPs and GSIFPs.

Target exon sequences are obtained from the Ensembl database (ensembl[dot]org). All sequences are double-checked using the UCSC database (genome[dot]ucsc[dot]edu) to ensure that all sequences are in the forward orientation. GSIFP design starts from the five nucleotides on the 5′ end of the exon sequences, while ensuring that the standard energies of GSIFPs in standard PCR conditions are between −11.5 kcal/mol and −12.5 kcal/mol at 60° C.

The possibility of forming primer dimers increases non-linearly as the number of different primers in a solution increases. Customized primer dimer checking software is used to check primer dimers. Primers that pass the primer dimer check are saved and considered “passed dimer primers.” Primers that fail the primer dimer check are redesigned until all primers pass the primer dimer check. The 3′ end of the GSOFPs are designed to start from the 5′ end of the GSIFPs. Primer design rules and primer dimer checks for GSOFPs are similar to those used for GSIFPs.

Once all GSIFPs and GSOFPs have passed the primer dimer check, the unique sequences starting from the 5′ end of the GSOFP and ending with the 3′ end of the GSIFP are BLASTR-ed (Altschul et al., J. Mol. Biol. 215:403-410 (1990)) against a human genome database to obtain only one exactly matched amplicon. Unique sequences that pass this step will then be double-checked against the UCSC database to ensure that only one amplicon can be found. Unique sequences that pass both of these steps are saved as the final designs for GSIFPs and GSOFPs.

If unique sequences fail either BLAST® check, they are redesigned until they pass the primer dimer check and both BLAST® checks.

### Example 2. Detecting RNA Fusion Using RRPs

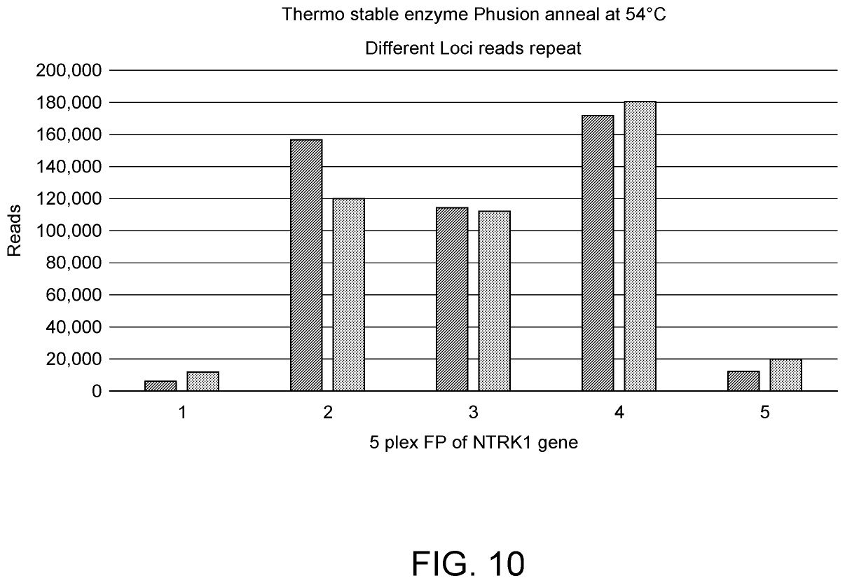

RNA fusion detection primers are designed to target the human genes NTRK1 and FGFR2 for potential gene fusions. First, a thermostable Phusion DNA polymerase is used at a 54° C., annealing temperature with a 72° C. extension following the protocol depicted in FIG. 2 to amplify gBlocks. Two experimental replicates are performed for five loci using GSIFPs and GSOFPs. For each locus, repeatability was good. See FIG. 10.

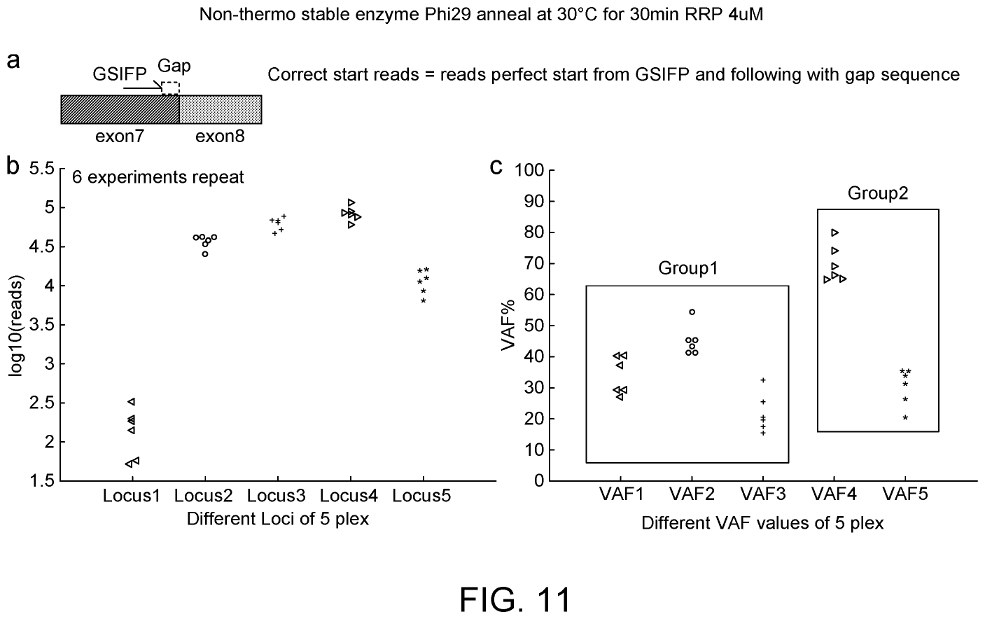

Next, the isothermal DNA polymerase phi29 was used following the protocol depicted in FIG. 3. phi29 was incubated at 30° C. for 30 minutes with an RRP concentration of 4 μM. The templates in this experiment were wildtype gBlocks (SEQ ID NOs: 15-17) mixed with equal amounts of different fusion gBlocks (SEQ ID NOs: 18-21). Results from this experiment are depicted in FIG. 11. Six experimental replicates are performed.

Correct Start Reads (CSR) were counted as reads that start perfectly from the first position of the GSIFP and continue 3′, including a gap sequence before reaching sequence from a second exon. See FIG. 11a. The system comprises five different loci with 5-plex IFP and OFP primers. Four of the five loci exhibited correct start reads within 2-amongst all six replicates. Three of the six replicates for one of the five loci are not considered to be repeatable since the CSR for the loci differ by over 2-fold.

The Variant Allele Frequency (VAF) value is defined as each fusion variant read divided by total reads of all fusion variants and WT reads for the same loci. FIG. 11c depicts two groups of VAF values which mean two loci. For each group, the sum of the VAF values is 100%. The same symbols show each of the six replicates. From the results, all the repeats of VAF values in the five tested loci are within 2-fold, which demonstrates the repeatability of the protocol.

### Example 3. Comparing Methods Using PCR and Isothermal Extension with RRPs

A NTRK1 gene panel was used to compare two different PCR protocols using gBlocks (e.g., SEQ ID NOs: 15-21) as templates.

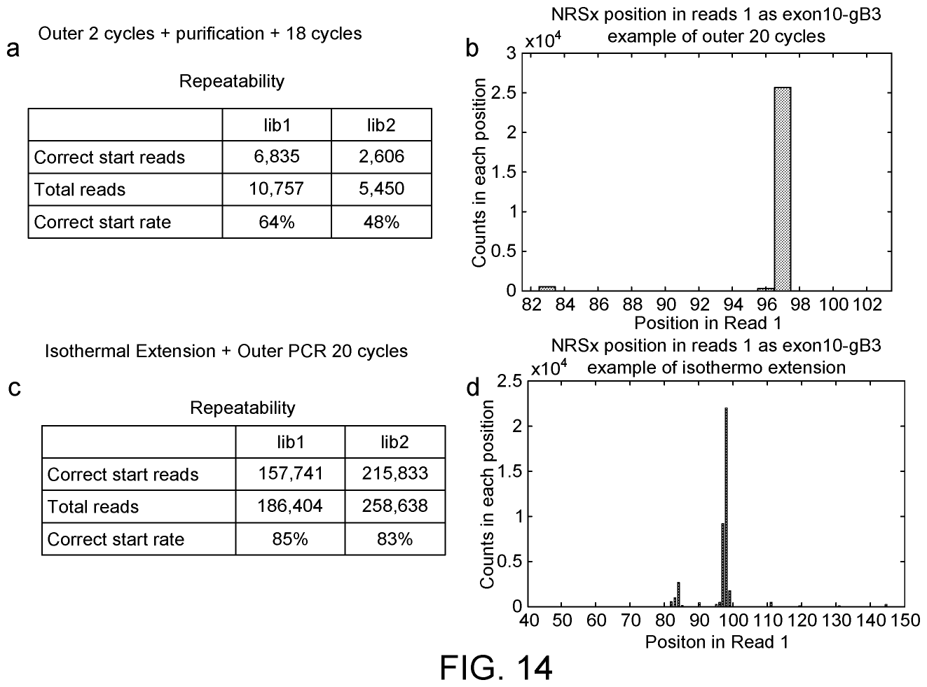

The first protocol comprised two cycles of PCR using Phusion HiFi DNA polymerase and using OFPs (e.g., SEQ ID NOs: 5-9) and RRPs, column purification, and then 18 cycles outer universal PCR, and two experimental replications (lib1 and lib2) were performed. The correct start rates (the percentage of the total reads that had a correct start position (e.g., correct start reads)) of the two experimental replications are showing in FIG. 14a. FIG. 14b shows the RRP binding sites in the gBlock 3. Only one domain binding position was observed using this protocol.

The second protocol comprised using the isothermal DNA polymerase phi29 DNA polymerase to perform one cycle of isothermal extension at 30° C. for one hour using OFPs (e.g., SEQ ID NOs: 5-9) followed by 20 cycles of outer PCR using ULOFPs and ULRPs with Phusion HiFi DNA polymerase. Two experimental replications (lib1 and lib2) were performed. The correct start rates are depicted in FIG. 4c. Notably, these values are higher than what was observed in the first protocol. In addition, more RRP binding positions were observed using the second protocol as compared to the first protocol. See FIG. 14d.

1. A method of preparing a sequencing library, the method comprising:
(a) introducing to a sample comprising at least one nucleic acid template molecule:
(i) at least one Randomer Reverse Primer (RRP), wherein the at least one RRP comprises a Randomer Sequence (RS) positioned within the 3′ region of the at least one RRP and a first universal adapter sequence positioned 5′ to the RS;
(ii) a first polymerase; and
(iii) a reaction buffer
to create a first mixture;
(b) subjecting the first mixture to a first polymerase extension step to generate a second mixture comprising at least one extended product;
(c) introducing to the second mixture:
(i) at least one Inner Forward Primer (IFP) that binds to an IFP binding site on the at least one nucleic acid template molecule;
(ii) a second adapter sequence;
(iii) a thermostable polymerase; and
(iv) one or more reagents for thermostable polymerase activity to generate a third mixture; and
(d) subjecting the third mixture to a second polymerase extension step to generate at least one amplicon of the at least one nucleic acid template molecule.

2. A method of preparing a sequencing library, the method comprising:
(a) introducing to a sample comprising at least one nucleic acid template molecule:
(i) at least one Randomer Reverse Primer (RRP), wherein the at least one RRP comprises a Randomer Sequence (RS) positioned on the 3′ region of the at least one RRP and a first universal adapter sequence positioned 5′ to the RS;
(ii) at least one Outer Forward Primer (OFP);
(iii) a first polymerase; and
(iv) a reaction buffer
to create a first mixture;
(b) subjecting the first mixture to a first polymerase extension step to generate a second mixture comprising at least one extended product;
(c) introducing to the second mixture:
(i) at least one Inner Forward Primer (IFP) that binds to an IFP binding site on the at least one nucleic acid template molecule;
(ii) a second adapter sequence;
(iii) a thermostable polymerase, and
(iv) one or more reagents for thermostable polymerase activity to generate a third mixture; and
(d) subjecting the third mixture to a second polymerase extension step to generate at least one amplicon of the at least one nucleic acid template molecule.

3. The method of claim 1, wherein the first polymerase extension step comprises isothermal extension.

4. The method of claim 1, wherein the method further comprises introducing to the sample at least one Outer Forward Primer (OFP) in step (a).

5. The method of claim 2, wherein the first polymerase extension step comprises thermal cycling.

6. The method of claim 1, wherein the second mixture is diluted between step (b) and step (c).

7. The method of claim 6, wherein the second mixture is diluted at a ratio between 1:10 and 1:10,000.

8. The method of claim 1, wherein the at least one extended product is purified from the second mixture between step (b) and step (c).

9. The method of claim 8, wherein the at least one extended product is purified using a technique selected from the group consisting of column purification and beads purification.

10. The method of claim 1, wherein the first universal adapter sequence is positioned at the 5′ end of the RRP.

11. The method of claim 1, wherein the RS comprises at least two degenerate nucleotides selected from the group consisting of R, Y, S, W, K, M, B, D, H, V, and N.

12. The method of claim 1, wherein the first universal adapter sequence is an Adapter_2 sequence.

13. The method of claim 1, wherein the second adapter sequence is an Adapter_3 sequence.

14. The method of claim 1, wherein the at least one RRP further comprises a Non-Relevant Sequence (NRS).

15. The method of claim 14, wherein the NRS comprises less than 50% complementarity with the at least one nucleic acid template molecule.

16. The method of claim 14, wherein the NRS comprises less than 50% complementarity to the first universal adapter sequence, the second adapter sequence, or both.

17. The method of claim 14, wherein the NRS comprises between 5 nucleotides and 20 nucleotides.

18. The method of claim 14, wherein the NRS is positioned 5′ to the RS and 3′ to the first universal adapter sequence.

19. The method of claim 1, wherein the RS comprises between 3 nucleotides and 30 nucleotides.

20. The method of claim 1, wherein the at least one RRP further comprises a Mixture of Specific Sequences (MSS) at the 3′ end of the at least one RRP.

21. The method of claim 20, wherein the MSS comprises between 2 nucleotides and 5 nucleotides.

22. The method of claim 20, wherein the MSS is comprised of A, T, G, and/or C nucleotides.

23. The method of claim 1, wherein the at least one RRP further comprises a Unique Molecular Identifier (UMI) sequence.

24. The method of claim 23, wherein the UMI sequence is positioned 5′ to the RS sequence and 3′ to the first universal adapter sequence.

25. The method of claim 23, wherein the UMI sequence comprises a mixture of between 10 and 100 defined DNA sequences with a minimum pairwise Hamming distance of between 2 and 5.

26. The method of claim 23, wherein the UMI sequence comprises a mixture of between 10 and 1000 defined DNA sequences with a minimum pairwise Levenschtein distance of between 2 and 5.

27. The method of claim 23, wherein the UMI sequence comprises at least one degenerate nucleotide selected from the group consisting of R, Y, S, W, K, M, B, D, H, V, and N.

28. The method of claim 1, wherein the at least one RRP further comprises a Sample Barcode (SB) sequence.

29. The method of claim 28, wherein the SB sequence is positioned 5′ to the RS sequence and 3′ to the first universal adapter sequence.

30. The method of claim 1, wherein the at least one nucleic acid template molecule is a DNA template molecule.

31. The method of claim 1, wherein the at least one nucleic acid template molecule is an RNA template molecule.

32. The method of claim 1, wherein the first polymerase, the thermostable polymerase, or both, is an RNA polymerase.

33. The method of claim 1, wherein the first polymerase, the thermostable polymerase, or both, is a DNA polymerase.

34. The method of claim 1, wherein the first polymerase is selected from the group consisting of phi29 DNA polymerase, DNA polymerase 1, large (Klenow) fragment, Klenow fragment, Bst DNA polymerase, T4 DNA polymerase, T7 DNA polymerase, Taq polymerase, Phusion® polymerase, Q5® polymerase, KAPA HiFi polymerase, Vent® DNA polymerase, LongAmp® Taq DNA polymerase, and OneTaq® DNA polymerase.

35. The method of claim 1, wherein the first polymerase, the thermostable polymerase, or both, is selected from the group consisting of Taq polymerase, Phusion® polymerase, Q5® polymerase, KAPA HiFi polymerase, Vent® DNA polymerase, LongAmp® Taq DNA polymerase, and OneTaq® DNA polymerase.

36. The method of claim 1, wherein the first polymerase is selected from the group consisting of a phi29 DNA polymerase, a Bst DNA polymerase, a Bst 2.0 DNA polymerase, a Bst 3.0 DNA polymerase, a T7 DNA polymerase, a T4 DNA polymerase, and a DNA polymerase 1 large (Klenow) fragment.

37. The method of claim 1, wherein the thermostable polymerase is selected from the group consisting of Taq polymerase, Phusion® polymerase, Q5® polymerase, KAPA HiFi polymerase, Vent® DNA polymerase, LongAmp® Taq DNA polymerase, and OneTaq® DNA polymerase.

38. The method of claim 3, wherein the isothermal extension occurs at a temperature between 20° C., and 65° C.

39. The method of claim 1, wherein the first polymerase extension step comprises a duration of between 10 seconds and 6 hours.

40. The method of claim 2, wherein the first polymerase extension step comprises thermal cycling.

41. The method of claim 40, wherein the thermal cycling comprises an annealing temperature of between 45° C., and 72° C.

42. The method of claim 1, wherein the second polymerase extension step comprises thermal cycling, and wherein the thermal cycling comprises an annealing temperature of between 45° C., and 72° C.

43. The method of claim 40, wherein the thermal cycling comprises between 1 and 50 thermal cycles.

44. The method of claim 2, wherein the method further comprises the introduction of at least one Universal Long Outer Forward Primer (ULOFP), at least one Universal Long Reverse Primer (ULRP), or both.

45. The method of claim 2, wherein the at least one OFP comprises a Gene Specific Outer Forward Primer (GSOFP) that binds to a GSOFP binding site on the at least one template nucleic acid molecule, and a second universal adapter sequence.

46. The method of claim 45, wherein the at least one ULOFP comprises at least 70% homology to the second universal adapter sequence.

47. The method of claim 44, wherein the at least one ULRP comprises at least 70% homology to the first universal adapter sequence.

48. The method of claim 44, wherein the at least one ULOFP comprises between 30 nucleotides and 100 nucleotides.

49. The method of claim 44, wherein the at least one ULRP comprises between 30 nucleotides and 100 nucleotides.

50. The method of claim 45, wherein the GSOFP comprises between 10 nucleotides and 70 nucleotides.

51. The method of claim 1, wherein the at least one amplicon is purified from the third mixture.

52. The method of claim 51, wherein the at least one amplicon is purified from the third mixture using column purification or beads purification.

53. The method of claim 1, wherein the at least one amplicon is diluted at a ratio of between 1:10 and 1:10,000.

54. The method of claim 1, wherein the at least one IFP comprises between 10 nucleotides and 70 nucleotides.

55. The method of claim 1, wherein the at least one IFP anneals to the at least one template nucleic acid molecule at a temperature between 45° C., and 72° C.

56. The method of claim 1, wherein the at least one IFP comprises a single-stranded sequence at its 5′ end that does not bind to the at least one template nucleic acid molecule.

57. The method of claim 56, wherein the single-stranded sequence comprises a sequencing adapter.

58. The method of claim 57, wherein the sequencing adapter is an adapter for adding an index adapter.

59. The method of claim 57, wherein the sequencing adapter is selected from the group consisting of an Illumina sequencing adapter, a Nanopore sequencing adapter, and an Ion Torrent sequencing adapter.

60. The method of claim 45, wherein the IFP binding site is positioned 5′ on the at least one nucleic acid template molecule as compared to the GSOFP binding site.

61. The method of claim 1, wherein the at least one IFP comprises at least one Gene Specific Inner Forward Primer (GSIFP) sequence, wherein the GSIFP sequence binds to the at least one template nucleic acid molecule at a GSIFP binding site.

62. The method of claim 45, wherein the IFP binding site and the GSOFP binding site overlap on the 5′ end of the IFP binding site and the 3′ end of the GSOFP binding site.

63. The method of claim 62, wherein the overlap comprises between 1 nucleotide and 40 nucleotides.

64. The method of claim 45, wherein between 1 nucleotide and 50 nucleotides are positioned between the 3′ end of the GSOFP binding site and the 5′ end of the IFP binding site.

65. The method of claim 64, wherein the IFP binding site is a GSIFP binding site.

66. The method of claim 45, wherein the IFP binding site and the GSOFP binding site are adjacent.

67. The method of claim 66, wherein the IFP binding site is a GSIFP binding site.

68. The method of claim 1, wherein the at least one OFP and at least one IFP provide a nested PCR that further comprises a middle PCR to improve the specificity and on-target rate.

69. The method of claim 68, wherein the middle PCR comprises using a Middle Forward Primer (MFP) that binds to an MFP binding site on the at least one template nucleic acid molecule, and wherein the MFP binding site partially overlaps with the OFP binding site, the IFP binding site, or both.

70. The method of claim 68, wherein the middle PCR comprises using a Middle Forward Primer (MFP) that comprises a 5′ region starting from the second nucleotide of the OFP 5′ region to the second nucleotide of the OFP 3′ region, and the MFP comprises a 3′ region starting from the second nucleotide of the IFP 5′ region to the second nucleotide of the IFP 3′ region.

71. The method of claim 69, wherein the MFP comprises between 10 nucleotides and 70 nucleotides.

72. The method of claim 1, wherein at least one template molecule comprises at least part of one exon, at least part of one intron, or both.

73. The method of claim 1, wherein the at least one template molecule comprises at least one long region of interest having a length of at least 50 nucleotides.

74. The method of claim 1, wherein the method comprises the use of a plurality of IFPs and a plurality of OFPs.

75. The method of claim 73, wherein a plurality of IFPs are used to tile the long region of interest.

76. The method of claim 75, wherein the tile is conducted in two orientations, one of which is based on the positive strand of the template nucleic acid molecule, and the other of which is based on the negative strand of the template nucleic acid molecule.

77. The method of claim 1, wherein the IFP binding site is positioned between 0 nucleotides and 20 nucleotides from a breakpoint of a gene fusion.

78. The method of claim 74, wherein the method comprises a first set of primers comprising a first OFP and a first IFP and a second set of primers comprising a second OFP and a second IFP, and wherein between 0 nucleotides and 100 nucleotides are positioned between the first set of primers and the second set of primers.

79. The method of claim 1, wherein the thermal cycling further comprises the use of at least one wildtype-specific blocker.

80. The method of claim 79, wherein the at least one wildtype-specific blocker corresponds to a wildtype sequence that overlaps with the at least one IFP by between 2 nucleotides and 30 nucleotides.

81. The method of claim 80, wherein:
(a) the overlap comprises a standard free energy of binding between −2 kcal/mol and −4 kcal/mol;
(b) sequence of the at least one IFP that does not overlap with the at least one wildtype-specific blocker comprises a standard free energy between −5 kcal/mol and −9 kcal/mol; and
(c) sequence of the at least one wildtype-specific blocker that does not overlap with the at least one IFP comprises a standard free energy between −7 kcal/mol and −12 kcal/mol.

82. The method of claim 79, wherein the at least one wildtype-specific blocker comprises a terminator to prevent 3′ to 5′ DNA polymerase exonuclease activity.

83. The method of claim 82, wherein the terminator is selected from the group consisting of a C3 spacer and DXXDM.

84. The method of claim 1, wherein the at least one or at least two IFPs are present at a concentration of between 1 nM and 1000 nM.

85. The method of claim 1, wherein the at least one or at least two OFPs are present at a concentration of between 1 nM and 1000 nM.

86. A method for detecting at least one gene fusion in a test sample, the method comprising:
(a) obtaining DNA extracted from a cell line or a clinical patient sample, or obtaining cDNA generated from RNA extracted from a cell line or a clinical patent sample to generate the test sample;
(b) introducing to the test sample:
(i) at least one Randomer Reverse Primer (RRP), wherein the at least one RRP comprises a Randomer Sequence (RS) positioned on the 3′ end of the at least one RRP and a first universal adapter sequence positioned 5′ to the RS;
(ii) at least one Outer Forward Primer (OFP) that binds to an OFP binding site;
(iii) a first polymerase; and
(iv) a reaction buffer
to create a first mixture;
(c) subjecting the first mixture to a first polymerase extension step to generate at least one extended product;
(d) diluting or purifying the at least one extended product to generate a second mixture comprising the at least one extended product;
(e) introducing to the second mixture
(i) at least one Inner Forward Primer (IFP) that binds to an IFP binding site;
(ii) a universal reverse primer;
(iii) a thermostable polymerase; and
(iv) one or more reagents for thermostable polymerase activity to generate a third mixture;
(f) subjecting the third mixture to a second polymerase extension step to generate at least one amplicon from the at least one extended product; and
(g) analyzing the at least one amplicon to identify an amplicon comprising the at least one gene fusion.

87. The method of claim 86, wherein the test sample comprises at least one gene fusion.

88. The method of claim 86, wherein the test sample does not comprise a gene fusion.

89. The method of claim 86, wherein the IFP binding site, the OFP binding site, or both, is on the positive strand of the DNA or cDNA.

90. The method of claim 86, wherein the IFP binding site, the OFP binding site, or both, is on the negative strand of the DNA or cDNA.

91. The method of claim 86, wherein the at least one amplicon comprises a target exon sequence.

92. The method of claim 91, wherein the 3′ end of the at least one IFP binds to an IFP binding site which is between 0 nucleotides and 20 nucleotides from the 3′ end of a target exon sequence for detecting a gene fusion.

93. The method of claim 86, wherein the method comprises the use of a first IFP and a second IFP, and wherein between 0 nucleotides and 100 nucleotides are positioned between the 3′ end of the first IFP and the 5′ end of the second IFP.

94. The method of claim 86, wherein the at least one IFP, the at least one OFP, or both, comprises a length of between 10 nucleotides and 100 nucleotides.

95. The method of claim 86, wherein the at least one IFP, the at least one OFP, or both, comprises a standard free energy between −11.5 kcal/mol and −12.5 kcal/mol in a standard PCR buffer.

96. The method of claim 86, wherein the at least one IFP, the at least one OFP, or both, does not form primer dimers.

97. The method of claim 86, wherein the concentration of the at least one IFP, at least one OFP, or both, is between 0.1 nM and 1000 nM.

98. The method of claim 86, wherein the total concentration of all OFPs and IFPs is less than 10 μM.

99. The method of claim 86, wherein step (g) comprises generating sequencing reads of the at least one amplicon using next generation sequencing.

100. The method of claim 99, wherein the method further comprises repeating steps (a) to (g) after adjusting the concentration of the at least one IFP to IFP_{new}, wherein IFP_{new}=IFP_{old}*(Reads_median/Reads_amplicon)^{X}, wherein IFP_{old} is the concentration of the at least one IFP in the first iteration of step (e); Reads_median is the median reads mapped to each amplicon; Reads_amplicon is the reads mapped to the amplicon corresponding to said forward primer; and X is an adjustment factor between 0.25 and 1.

101. A method of designing a plurality of Inner Forward Primers (IFPs) and a plurality of Outer Forward Primers (OFPs) to identify a gene fusion, within the gene of interest, wherein an adjacent IFP and OFP designed to amplify the same target region of the gene of interest form a primer set, and wherein:
(a) the 3′ end of an OFP binding site and the 5′ end of an IFP binding site overlap for a primer set;
(b) at least one nucleotide is positioned between the 3′ end of an OFP binding site and the 5′ end of an IFP binding site for a primer set; or
(c) zero nucleotides are positioned between the 3′ end of an OFP binding site and the 5′ end of the IFP binding site, and the OFP binding site and an IFP binding site do not overlap for a primer set;
wherein the IFPs and OFPs are used to identify a gene fusion in the nucleic acid molecule.

102. The method of claim 101, wherein the IFPs, the OFPs, or both, are designed to hybridize to the positive strand of a reference sequence of the nucleic acid molecule.

103. The method of claim 101, wherein the IFPs, the OFPs, or both, are designed to hybridize to the negative strand of a reference sequence of the nucleic acid molecule.

104. The method of claim 101, wherein the IFPs, the OFPs, or both, comprise a length of between 10 nucleotides and 100 nucleotides.

105. The method of claim 101, wherein the IFPs, the OFPs, or both, comprise a standard free energy between −11.5 kcal/mol and −12.5 kcal/mol in a standard PCR buffer.

106. The method of claim 101, wherein the IFPs, the OFPs, or both, do not form primer dimers.

107. The method of claim 101, wherein the method comprises designing IFPs, OFPs, or both, for the forward strand and the reverse strand of the nucleic acid molecule.

108. The method of claim 101, wherein the IFPs enable identification of all exons involved in the gene fusion.

109. The method of claim 101, wherein the IFPs enable identification of all introns involved in the gene fusion.

110. The method of claim 101, wherein the nucleic acid molecule is a DNA molecule.

111. The method of claim 101, wherein the nucleic acid molecule is an RNA molecule.

112. The method of claim 101, wherein the IFPs and OFPs tile an entire intron region of the gene fusion.

113. The method of claim 101, wherein the IFPs and OFPs tile an entire exon region of the gene fusion.

114. The method of claim 101, wherein the method further comprises generating at least one amplicon of the nucleic acid molecule using the OFPs and IFPs via isothermal extension, PCR, or both.

115. A method for detecting alternative RNA splicing, the method comprising:
(a) introducing to a sample comprising cDNA generated from an RNA sample comprising at least one RNA splicing variant:
(i) at least one Randomer Reverse Primer (RRP), wherein the at least one RRP comprises a Randomer Sequence (RS) positioned on the 3′ end of the at least one RRP and a first universal adapter sequence positioned 5′ to the RS;
(ii) a first polymerase; and
(iii) a reaction buffer
to create a first mixture;
(b) subjecting the first mixture to a first polymerase extension step to generate a second mixture comprising at least one extended product;
(c) purifying the at least one extended product from step (b) to generate a second mixture comprising the at least one extended product;
(d) introducing to the second mixture:
(i) at least one Inner Forward Primer (IFP) that binds to an IFP binding site on the at least one nucleic acid template molecule;
(ii) a second adapter sequence;
(iii) a thermostable polymerase; and
(iv) one or more reagents for thermostable polymerase activity to generate a third mixture;
(e) subjecting the third mixture to thermal cycling to generate at least one amplicon of the at least one extended product; and
(f) analyzing the at least one amplicon to identify the at least one RNA splicing variant.

116. The method of claim 115, wherein the at least one OFP targets at least one exon of a gene comprising at least one RNA splicing variant.

117. The method of claim 115, wherein the at least one OFP targets every exon of a gene comprising the at least one RNA splicing variant.

118. The method of 115, wherein the at least one amplicon is purified following step (f).

119. The method of claim 118, wherein an index primer for sequencing is added to the at least one amplicon.

120. The method of claim 119, wherein the method further comprises sequencing the at least one amplicon using a sequencing instrument selected from the group consisting of an Oxford Nanopore sequencer, a PacBio sequencer, an Illumina Miseq sequencer, an Illumina MiniSeq sequencer, an Illumina NextSeq sequencer, an Ion Torrent sequencer, and an Illumina Hiseq sequencer to generate at least one sequencing read.

121. The method of claim 120, wherein the at least one sequencing read is aligned to a reference sequence to identify the at least one RNA splicing variant.


SEQUENCE LISTING (57 sequences)

SEQ ID NO: 1 - DNA, 52 bp, synthetic construct
  source 1..52: mol_type = other DNA
gtctcgtgggctcggagatgtgtataagagacaggtgcaagnnnnnnnnnnn

SEQ ID NO: 2 - DNA, 34 bp, synthetic construct
  source 1..34: mol_type = other DNA
gtctcgtgggctcggagatgtgtataagagacag

SEQ ID NO: 3 - DNA, 18 bp, synthetic construct
  source 1..18: mol_type = other DNA
cgtgggctcggagatgtg

SEQ ID NO: 4 - DNA, 44 bp, synthetic construct
  source 1..44: mol_type = other DNA
tgcgatgcaatgagaacattagaagtcagctgcatgactgaaga

SEQ ID NO: 5 - DNA, 61 bp, synthetic construct
  source 1..61: mol_type = other DNA
tgcgatgcaatgagaacattagaagtcagctgcatgactgaagaggggatgcaccagtggt

SEQ ID NO: 6 - DNA, 67 bp, synthetic construct
  source 1..67: mol_type = other DNA
tgcgatgcaatgagaacattagaagtcagctgcatgactgaagaaaaggtgtttcgtccttcttctc

SEQ ID NO: 7 - DNA, 65 bp, synthetic construct
  source 1..65: mol_type = other DNA
tgcgatgcaatgagaacattagaagtcagctgcatgactgaagaaaaggaagaggcaggcaaaga

SEQ ID NO: 8 - DNA, 66 bp, synthetic construct
  source 1..66: mol_type = other DNA
tgcgatgcaatgagaacattagaagtcagctgcatgactgaagatgtcatgaaatgcagggacatg

SEQ ID NO: 9 - DNA, 62 bp, synthetic construct
  source 1..62: mol_type = other DNA
tgcgatgcaatgagaacattagaagtcagctgcatgactgaagaagctcccacttgagcacg

SEQ ID NO: 10 - DNA, 49 bp, synthetic construct
  source 1..49: mol_type = other DNA
tcgtcggcagcgtcagatgtgtataagagacagccaccgccgtgtgcag

SEQ ID NO: 11 - DNA, 51 bp, synthetic construct
  source 1..51: mol_type = other DNA
tcgtcggcagcgtcagatgtgtataagagacagccgggtctccagatgtgc

SEQ ID NO: 12 - DNA, 48 bp, synthetic construct
  source 1..48: mol_type = other DNA
tcgtcggcagcgtcagatgtgtataagagacagggccaggcccacagc

SEQ ID NO: 13 - DNA, 51 bp, synthetic construct
  source 1..51: mol_type = other DNA
tcgtcggcagcgtcagatgtgtataagagacagcccatcctctggagccag

SEQ ID NO: 14 - DNA, 50 bp, synthetic construct
  source 1..50: mol_type = other DNA
tcgtcggcagcgtcagatgtgtataagagacaggtcccggcgcttgatgt

SEQ ID NO: 15 - DNA, 500 bp, synthetic construct
  source 1..500: mol_type = other DNA
cttcaaggcagagcaaatgtgacactgtatcagtactggaagaagacattcatacattaaggcatggaagtgaataagcgggccgggcagaggtctctgttcaggtcaacgtctccttcccggccagtgtgcagctgcacacggcggtggagatgcaccactggtgcatccccttctctgtggatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgacactaacagcacatctggagacccggtggagaagaaggacgaaacaccttttggg

SEQ ID NO: 16 - DNA, 500 bp, synthetic construct
  source 1..500: mol_type = other DNA
cttcaaggcagagcaaatgtgacactgtatcagtactggaagaagacattcatacattaaggcatggaagtgaataagcgatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaacc

SEQ ID NO: 17 - DNA, 500 bp, synthetic construct
  source 1..500: mol_type = other DNA
cttcaaggcagagcaaatgtgacactgtatcagtactggaagaagacattcatacattaaggcatggaagtgaataagcgggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaag

SEQ ID NO: 18 - DNA, 494 bp, synthetic construct
  source 1..494: mol_type = other DNA
cttcaaggcagagcaaatgtgacactgtatcagtactggaagaagacattcatacattaaggcatggaagtgaataagcgcatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtgagtcactggaactggaggacccgtcttctgggctgggtgtgaccaagcaggatctgggcccagacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggct

SEQ ID NO: 19 - DNA, 499 bp, synthetic construct
  source 1..499: mol_type = other DNA
cttcaaggcagagcaaatgtgacactgtatcagtactggaagaagacattcatacattaaggcatggaagtgaataagcgttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaaggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagt

SEQ ID NO: 20 - DNA, 496 bp, synthetic construct
  source 1..496: mol_type = other DNA
cttcaaggcagagcaaatgtgacactgtatcagtactggaagaagacattcatacattaaggcatggaagtgaataagcgctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacgtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagt

SEQ ID NO: 21 - DNA, 492 bp, synthetic construct
  source 1..492: mol_type = other DNA
cttcaaggcagagcaaatgtgacactgtatcagtactggaagaagacattcatacattaaggcatggaagtgaataagcgctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaac

SEQ ID NO: 22 - DNA, 53 bp, synthetic construct
  source 1..53: mol_type = other DNA
tcgtcggcagcgtcagatgtgtataagagacaggccattcaaggtaatcgccg

SEQ ID NO: 23 - DNA, 59 bp, synthetic construct
  source 1..59: mol_type = other DNA
tcgtcggcagcgtcagatgtgtataagagacagcttcagtttagttgaggataccacat

SEQ ID NO: 24 - DNA, 58 bp, synthetic construct
  source 1..58: mol_type = other DNA
tcgtcggcagcgtcagatgtgtataagagacaggaaacttggtacttcatggtgaatg

SEQ ID NO: 25 - DNA, 60 bp, synthetic construct
  source 1..60: mol_type = other DNA
tcgtcggcagcgtcagatgtgtataagagacaggaagattttgtcagtgagaacagtaac

SEQ ID NO: 26 - DNA, 52 bp, synthetic construct
  source 1..52: mol_type = other DNA
tcgtcggcagcgtcagatgtgtataagagacagcaggagcatcgcattggag

SEQ ID NO: 27 - DNA, 55 bp, synthetic construct
  source 1..55: mol_type = other DNA
tcgtcggcagcgtcagatgtgtataagagacagcaatcacacgtaccacctggat

SEQ ID NO: 28 - DNA, 51 bp, synthetic construct
  source 1..51: mol_type = other DNA
tcgtcggcagcgtcagatgtgtataagagacaggggctgccctacctcaag

SEQ ID NO: 29 - DNA, 54 bp, synthetic construct
  source 1..54: mol_type = other DNA
tcgtcggcagcgtcagatgtgtataagagacagctcactgtcctgccaaaacag

SEQ ID NO: 30 - DNA, 53 bp, synthetic construct
  source 1..53: mol_type = other DNA
tcgtcggcagcgtcagatgtgtataagagacagctctgcatggttgacagttc

SEQ ID NO: 31 - DNA, 52 bp, synthetic construct
  source 1..52: mol_type = other DNA
tcgtcggcagcgtcagatgtgtataagagacagccaaacgtatccccctgcg

SEQ ID NO: 32 - DNA, 55 bp, synthetic construct
  source 1..55: mol_type = other DNA
tcgtcggcagcgtcagatgtgtataagagacaggacccaaaatgggagtttccaa

SEQ ID NO: 33 - DNA, 51 bp, synthetic construct
  source 1..51: mol_type = other DNA
tcgtcggcagcgtcagatgtgtataagagacagaccgtggccgtgaagatg

SEQ ID NO: 34 - DNA, 56 bp, synthetic construct
  source 1..56: mol_type = other DNA
tcgtcggcagcgtcagatgtgtataagagacagataaatcttcttggagcctgcac

SEQ ID NO: 35 - DNA, 53 bp, synthetic construct
  source 1..53: mol_type = other DNA
tcgtcggcagcgtcagatgtgtataagagacagggcatggagtacttggcttc

SEQ ID NO: 36 - DNA, 64 bp, synthetic construct
  source 1..64: mol_type = other DNA
tcgtcggcagcgtcagatgtgtataagagacaggccagagatatcaacaatatagactattaca

SEQ ID NO: 37 - DNA, 62 bp, synthetic construct
  source 1..62: mol_type = other DNA
tcgtcggcagcgtcagatgtgtataagagacagcctgtttgatagagtatacactcatcaga

SEQ ID NO: 38 - DNA, 50 bp, synthetic construct
  source 1..50: mol_type = other DNA
tcgtcggcagcgtcagatgtgtataagagacaggccagccaactgcacca

SEQ ID NO: 39 - DNA, 57 bp, synthetic construct
  source 1..57: mol_type = other DNA
tcgtcggcagcgtcagatgtgtataagagacagggatcgaattctcactctcacaac

SEQ ID NO: 40 - DNA, 61 bp, synthetic construct
  source 1..61: mol_type = other DNA
tgcgatgcaatgagaacattagaagtcagctgcatgactgaagacggaggagcgttgccat

SEQ ID NO: 41 - DNA, 58 bp, synthetic construct
  source 1..58: mol_type = other DNA
tgcgatgcaatgagaacattagaagtcagctgcatgactgaagacctggcccggccct

SEQ ID NO: 42 - DNA, 67 bp, synthetic construct
  source 1..67: mol_type = other DNA
tgcgatgcaatgagaacattagaagtcagctgcatgactgaagatactgccagtaggactgtagaca

SEQ ID NO: 43 - DNA, 65 bp, synthetic construct
  source 1..65: mol_type = other DNA
tgcgatgcaatgagaacattagaagtcagctgcatgactgaagaatgaggatgacaccgatggtg

SEQ ID NO: 44 - DNA, 67 bp, synthetic construct
  source 1..67: mol_type = other DNA
tgcgatgcaatgagaacattagaagtcagctgcatgactgaagagctgaaaaacgggaaggagttta

SEQ ID NO: 45 - DNA, 68 bp, synthetic construct
  source 1..68: mol_type = other DNA
tgcgatgcaatgagaacattagaagtcagctgcatgactgaagatgtagtggagaatgaatacgggtc

SEQ ID NO: 46 - DNA, 68 bp, synthetic construct
  source 1..68: mol_type = other DNA
tgcgatgcaatgagaacattagaagtcagctgcatgactgaagaggaaaagaacggcagtaaatacgg

SEQ ID NO: 47 - DNA, 61 bp, synthetic construct
  source 1..61: mol_type = other DNA
tgcgatgcaatgagaacattagaagtcagctgcatgactgaagaaggccaaccagtctgcc

SEQ ID NO: 48 - DNA, 67 bp, synthetic construct
  source 1..67: mol_type = other DNA
tgcgatgcaatgagaacattagaagtcagctgcatgactgaagagtgcttggcgggtaattctattg

SEQ ID NO: 49 - DNA, 63 bp, synthetic construct
  source 1..63: mol_type = other DNA
tgcgatgcaatgagaacattagaagtcagctgcatgactgaagaagccagacttcagcagcca

SEQ ID NO: 50 - DNA, 67 bp, synthetic construct
  source 1..67: mol_type = other DNA
tgcgatgcaatgagaacattagaagtcagctgcatgactgaagagtctccgagtatgaacttccaga

SEQ ID NO: 51 - DNA, 61 bp, synthetic construct
  source 1..61: mol_type = other DNA
tgcgatgcaatgagaacattagaagtcagctgcatgactgaagaacaagcccaaggaggcg

SEQ ID NO: 52 - DNA, 73 bp, synthetic construct
  source 1..73: mol_type = other DNA
tgcgatgcaatgagaacattagaagtcagctgcatgactgaagaatgaagatgattgggaaacacaagaatat

SEQ ID NO: 53 - DNA, 64 bp, synthetic construct
  source 1..64: mol_type = other DNA
tgcgatgcaatgagaacattagaagtcagctgcatgactgaagatggtgtcatgcacctaccag

SEQ ID NO: 54 - DNA, 70 bp, synthetic construct
  source 1..70: mol_type = other DNA
tgcgatgcaatgagaacattagaagtcagctgcatgactgaagatgatgaaaatagcagactttggactc

SEQ ID NO: 55 - DNA, 64 bp, synthetic construct
  source 1..64: mol_type = other DNA
tgcgatgcaatgagaacattagaagtcagctgcatgactgaagaaagtggatggctccagaagc

SEQ ID NO: 56 - DNA, 69 bp, synthetic construct
  source 1..69: mol_type = other DNA
tgcgatgcaatgagaacattagaagtcagctgcatgactgaagactgaaggaaggacacagaatggata

SEQ ID NO: 57 - DNA, 67 bp, synthetic construct
  source 1..67: mol_type = other DNA
tgcgatgcaatgagaacattagaagtcagctgcatgactgaagacgttcaagcagttggtagaagac